(19) United States
(12) Patent Application Publication
(10) Pub. No.: US 2006/0184088 A1
(43) Pub. Date: Aug. 17, 2006

(54) Device and method providing arterial blood flow for perfusion of ischemic myocardium

(75) Inventors: Richard Van Bibber, Redmond, WA (US); Scott Wolf, Bellevue, WA (US); Clifton A. Alferness, Redmond, WA (US); John M. Adams, Sammamish, WA (US)

Correspondence Address: JOSEPH J. BASISTA, 8624 154TH AVE NE, REDMOND, WA, 98052, US

(73) Assignee: Scout Medical Technologies, LLC

(21) Appl. No.: 11/399,836

(22) Filed: Apr. 6, 2006

Related U.S. Application Data
(62) Division of application No. 10/236,386, filed on Sep. 6, 2002.
(60) Provisional application No. 60/388,005, filed on Jun. 11, 2002.

(51) Int. Cl. A61M 5/00

(52) U.S. Cl. 604/8

(57) ABSTRACT

The present invention generally relates to methods and apparatus for use in endovascular and intraoperative procedures providing arterial blood-flow for perfusion of ischemic myocardium. Aspects of the present invention provide a conduit between a non-coronary sinus of the aorta and a coronary vein. The conduit traverses a portion of the right atrium and the coronary sinus, and is located entirely within the heart and aorta. Arterial blood flows from the aorta through the conduit and into the coronary venous circulation towards the ischemic region of the heart. All procedures described herein may be performed endovascularly, and further may be performed while the patient's heart is beating.

### PRIORITY

This application is a divisional of co-pending application Ser. No. 10/236,386 filed Sep. 6, 2002, and further claims priority from Provisional Application No. 60/388,005 filed Jun. 11, 2002, entitled “Method and Apparatus for an Aorta to Atrium Anastomosis for Venous Retroperfusion of Ischemic Myocardium.”

### FIELD OF THE INVENTION

The present invention relates generally to methods and apparatus for treating ischemic heart disease. More particularly, the invention relates to endovascular devices and methods of providing arterial blood flow from the aorta to a portion of the coronary vascular system for perfusion of ischemic myocardium.

### BACKGROUND

Coronary artery disease (CAD), also known as ischemic heart disease, affects more than 12.5 million Americans according to the American Heart Association (AHA). CAD is the leading cause of death and disability in the United States, killing over half a million people in 1999. This is a progressive disease that causes narrowing of the arteries that supply blood to the heart muscle, thus diminishing cardiac perfusion. Eventually, the delivery of blood is not sufficient to maintain proper function of the heart. The most common manifestation of the disease is angina pectoris or chest pain, which can be severe. The AHA estimates that well over six million Americans suffer from angina pectoris, with over 400,000 new cases each year. However, complications that are even more serious can develop including myocardial infarction (heart attack), arrhythmia (irregular or lack of a heart beat), sudden death from cardiac arrest, and heart failure.

The cardiac perfusion system is composed of two coronary arterial vessels, the left and right coronary arteries, which perfuse the myocardium from the epicardial surface inward towards the endocardium. Perfused blood flows through the capillary systems, into the coronary veins, and then into the right atrium via the coronary sinus. Additional systems, such as the lymphatic and the Thebesian, also provide drainage pathways for coronary blood. The venous system has extensive collaterals and, unlike the coronary arteries, does not occlude in atherosclerotic disease.

Current options to treat CAD caused, for example by atherosclerosis, include medical therapy/lifestyle changes, percutaneous intervention such as percutaneous transluminal coronary angioplasty (PTCA) often with coronary stenting, and surgical intervention such as coronary artery bypass grafting (CABG). PTCA and CABG have emerged as the leading treatments for coronary artery disease when drug therapy and lifestyle modification fail or are inadequate. The goal of both types of treatment is to restore the flow of arterial blood through the arteries and down to the level of the microcirculation. These treatments have been highly successful in reducing or eliminating symptoms and improving the quality of life for those suffering.

Best known of the current surgical techniques is CABG, wherein a thoracotomy is performed to expose the patient's heart, and one or more blocked coronary arteries are bypassed with saphenous veins. In preparation for the bypass grafting, the heart is arrested using a suitable cardioplegia solution, while the patient is placed on cardiopulmonary bypass (i.e., a heart-lung machine) to maintain circulation throughout the body during the operation. Typically, a state of hypothermia is induced in the heart muscle during the bypass operation to reduce oxygen utilization, thereby preserving the tissue from further necrosis. Alternatively, the heart may be perfused throughout the operation using either normal or retrograde flow through the coronary sinus, with or without hypothermia. Once the bypass grafts are implanted, the heart is resuscitated, and the patient is removed from cardiopulmonary bypass. Drawbacks of conventional open heart surgery are that such surgery is time-consuming and costly, involves a significant risk of mortality, requires a lengthy period of recuperation, and involves significant discomfort to the patient.

As a result of the foregoing drawbacks to the above surgical techniques, other less invasive surgical techniques have been developed that permit coronary bypass grafting to be performed endoscopically, i.e., using elongated instruments inserted through incisions located between the ribs. A drawback of these keyhole techniques, however, is that they can be used only for coronary arteries that are readily accessible, and not, for example, those located posteriorly.

Alternatively, techniques such as PTCA have been developed for reopening arteries, such as the coronary arteries, that have become constricted by plaque. In these techniques, a balloon catheter is typically inserted into the stenosis and then inflated to compress and crack the plaque lining the vessel, thereby restoring patency to the vessel. Additionally, a vascular prosthesis, commonly referred to as a “stent,” may be inserted transvascularly and expanded within the vessel after the angioplasty procedure, to maintain the patency of the vessel.

A drawback of the foregoing transvascular approaches is that the treatment device, e.g., the balloon catheter or the stent delivery system must be inserted in the vessel before it can be expanded. Occasionally, a stenosis may occlude so much of a vessel that there is insufficient clearance to advance a guidewire and catheter within the stenosis to permit treatment. In addition, arterial blockages treatable using PTCA techniques are restricted to the portions of the anatomy where such techniques can be beneficially employed.

Moreover, the above-described technique-both open-surgery and transvascular-are useful only where the stenosis is localized, so that the bypass graft or PTCA procedure will restore near normal blood flow to the effected areas. Yet, current technology offers little relief or hope for a population of patients suffering from diffuse atherosclerosis where blockages exist throughout much of the coronary arterial system. Others in the population have, for example, extensive diffuse arterial disease with no good distal arterial target, persistent recurrent restenosis, or small vessels with no good target for arterial revascularization. Some of these patients may have already had one or more failed PTCA and CABG procedures. Some may be candidates for CABG but are excluded due to surgical risk and co-morbidity. For a large number of this patient population in the later phases of CAD, and particularly diffuse atherosclerotic disease, current technology offers little relief or hope. In such instances, humanely extending the patient's life for additional months may provide significant physical and emotional benefits for the patient.

Estimates of the size of this patient population vary, but several reports indicate it to be around 10% of those needing revascularization. Some of these patients may be considered for heart transplantation, though their numbers far exceed the supply of suitable hearts, and many patients could not tolerate such an invasive surgical procedure. Recently, some of these “no option” patients have been involved in a variety of new experimental therapies including trials of direct myocardial revascularization (DMR), percutaneous myocardial revascularization (PMR), gene or protein injections for angiogenesis, and coronary venous retroperfusion. Direct percutaneous myocardial revascularization and angiogenesis trials have met with mixed results. Some patients report feeling better, but the therapeutic benefits of these techniques have yet to be established. One criticism has been that the creation of new vasculature in the neighborhood of the microcirculation is ineffective because the problem lies upstream in the larger blocked arterial conduits. The arterial blood supply will still be limited by the stenosis or stenoses in the larger vessel or vessels.

The coronary veins are attractive as conduits to chronically deliver oxygenated blood to ischemic myocardium in patients with severe CAD. First, the atherosclerotic process that impairs the arteries virtually never affects the veins. Second, the coronary venous system is easily accessed via the coronary sinus, which is located in the right atrium. Third, a redundant drainage system (coronary sinus, Thebesian system, anterior cardiac veins) in the heart allows for retroperfusion and delivery of oxygen at the capillary level while still providing a means for draining blood. Lastly, ample experimental evidence and limited clinical evidence indicate that coronary venous retroperfusion can reduce or eliminate myocardial ischemia and angina due to impaired arterial inflow. It is also worthy to note that retroperfusion of the coronary sinus is considered a standard method to preserve myocardium during cardiopulmonary bypass. A procedure that could permanently bring arterial blood to the coronary venous system in a minimally invasive way has the potential to help improve the symptoms and quality of life of numerous CAD patients who currently have no proven alternatives.

Over the past several decades, surgeons have occasionally used a coronary vein as a means of oxygenating myocardium when a suitable arterial target could not be found. In many patients, the aorta-coronary vein bypass (CVBG) or internal mammary artery (IMA) to coronary vein bypass surgical procedures provides relief from angina. Follow up examination in some cases has shown open grafts several years after the surgery. Researchers working with surgical animal models have shown short-term and long-term benefit to coronary venous retroperfusion in the presence of arterial occlusion. Long-term graft patency and nutritive flow to the myocardium have been demonstrated. Recently, a percutaneous approach to retroperfusion has been successfully used in a small group of patients. In these people, a portion of a functioning coronary artery was connected to an adjacent coronary vein to provide blood flow for venous retroperfusion. Follow-up data indicate improvement in symptoms and persistent patency. With this documentation of safety and feasibility, there is now a foundation to explore additional endovascular approaches to cardiac venous retroperfusion.

Percutaneous approaches to coronary venous retroperfusion are being explored. An approach is to bring oxygenated blood from the left ventricle through the venous system to the ischemic myocardium. This approach requires creating holes or channels between coronary vessels and ventricular heart chambers. Other disadvantages of this approach are that the blood flowing from the left ventricle is out of phase with the normal cardiac arterial supply, the blood pressure is too high, and there is a tendency of the blood to flow back into the left ventricle during the relaxation phase. As a result, pressure limiting and back flow preventing valves must be implanted in an effort to approximate natural or normal blood flow. Another approach involves bringing oxygenated blood from a coronary artery that is adjacent or near the target vein. A significant disadvantages of this technique is encountered when a suitable vein does not lie in close proximity to the proximal end of the diseased segment of coronary artery.

In view of the foregoing, it would be desirable to provide methods and apparatus for endovascular implantation in a beating heart that provide arterial blood flow for venous retroperfusion to ischemic myocardium, particularly for the population of patients having few other options. It would further be desirable to provide methods and apparatus that enable patients suffering the later phases of diffuse ischemic heart disease to experience renewed vigor, reduced pain, and improved emotional well being during the remainder of their lives.

### SUMMARY

An embodiment of the present invention includes a device that provides arterial blood flow from aorta to coronary venous system for venous retroperfusion of myocardium. The device includes an aorta-right atrium traversing connector arranged to receive arterial blood flow from the aorta, an arterial blood conduit in fluid communication with the traversing connector and a portion of the venous system, the conduit arranged for placement within the right atrium and the coronary sinus, and a venous connector arranged to couple the conduit with the coronary venous system. The aorta-right atrium traversing connector may include an inlet member arranged for receiving arterial blood flow from the aorta and for traversing a first aperture in an aortic wall and a second aperture in a right atrium wall, and having a channel providing fluid communication.

The arterial blood conduit may include a tubular member having a first end, a second end, and a lumen providing fluid communication between the ends, the tubular material comprising a flexible material. The arterial blood conduit may further include a member having a first end adapted to be coupled to the aorta-right atrium traversing connector, a second end adapted to be coupled to the venous connector, an intermediate portion located between the ends, a lumen providing fluid communication between the ends, a first region near the first end adapted to be placed in the right atrium and a second region near the second end adapted to be placed into a portion of the venous system, the member comprising a flexible-material. The intermediate portion of conduit may include a self-sealing diaphragm. The conduit may include a biocompatible material that comprises at least one from the group consisting of polyvinyl chloride, polyethylene, polytetrafluoroethylene (PTFE), and ePTFE.

The venous connector may include a radially expandable elongated structure that includes a portion arranged for annular enlargement and configured for disposition around the inside of a lumen of the coronary venous system, and which, when annularly enlarged within the lumen, engages the conduit with the vascular lumen. The device may include arrangement for endovascular implantation, which may further be in a beating heart.

The invention further provides an aorta-right atrium traversing connector. The connector includes an inlet member arranged for receiving arterial blood flow from the aorta and for traversing a first aperture in an aortic wall and a second aperture in a right atrium wall, and having a channel providing fluid communication. The first aperture may occur at a point proximate to a non-coronary aortic sinus. The inlet member may include arrangement for coupling with a conduit arranged to carry the arterial blood flow. The inlet member may further include an annularly enlargeable structure that, when annularly enlarged within a portion of a conduit arranged to carry the arterial blood flow, couples the inlet member to the conduit. The inlet member may include arrangement to move from a first configuration for endovascular placement in the first and second apertures to a second configuration of implantation in the first and second apertures. A portion of the inlet member may include arrangement for self-annular expansion after deployment from a sheath. A portion of the inlet member may further include arrangement for annular enlargement by expansion of an inflatable expandable structure positioned within the portion of the inlet member. The inlet member may include at least one element that extends radially outward and arranged to engage an interior portion of the aortic wall. The channel may include a portion of arterial blood conduit arranged around a portion of the inlet member. The connector may include arrangement for endovascular implantation, which may be in a beating heart.

The invention still further provides an aorta-right atrium traversing connector. The traversing connector includes an inlet member arranged for receiving arterial blood flow from the aorta and for traversing a first aperture in an aortic wall and a second aperture in a right atrium wall, and having a channel providing fluid communication, and a positioning member arranged to maintain the inlet member in a selected position. The inlet member may include arrangement for engaging the aorta. The positioning member may include an element for engaging an interior wall of the right atrium, and may include arrangement for engaging the right atrium and the inlet member. The positioning member may include at least one element extending radially outward, and arranged to engage an interior portion of the right-atrial wall and position the inlet member relative to the right-atrial wall. The radially extending element may include arrangement for moving from a first configuration for endovascular placement to a second configuration for engagement. A portion of the positioning member may include arrangement to resist annular enlargement.

The inlet member may include an element for engaging an aortic interior wall, and the positioning member may include an element for engaging a right-atrial interior wall, and when a portion of the positioning member engages a portion of the inlet member, the inlet member engaging element and the position member engaging element are arranged to cooperatively compress tissue radial of the apertures between them. The compression may limit blood leakage from at least one of the aorta and the right atrium.

The invention also provides an assembly for use in creating a guidewire pathway between two body structures each having a cavity. The assembly includes a first catheter having a distal tip arranged for placement into a cavity of a body structure and a lumen, a second catheter having a distal tip arranged for placement into a cavity of another body structure and a lumen, and a tissue penetrating element deployable from one lumen and arranged to create a guidewire pathway by penetrating tissue. The cavity of a body structure may include a lumen of a vascular structure, or may include a cardiac chamber. One catheter may include arrangement for transvascular placement in an arterial structure, or for transvascular placement in a venous structure. Alternatively, one catheter may include arrangement for transvascular placement in an arterial structure and another catheter may include arrangement for transvascular placement in a venous structure. One distal tip may carry a magnetic member arranged to attract and align with a magnetic member carried on another distal tip. One distal tip may carry an electrical signal source and another distal tip may carry an electrical signal sensor. One distal tip may carry an ultrasound source and another distal tip may carry an ultrasound sensor. One distal tip may carry a light source and another distal tip may carry a light sensor. One distal tip may include a substance viewable with an imaging device. Further, one catheter may be arranged to deploy the penetrating element, and another may be arranged to engage the penetrating element when the penetrating element is deployed from another catheter. One catheter may be arranged to deploy the penetrating element, and another catheter may further include member arranged to snare the penetrating element. One catheter may include an additional lumen arranged to eject a substance viewable with an imaging device.

The penetrating element may be carried on a guidewire. The penetrating element may include a penetration aid selected from a group consisting of a thermal heating element, a laser energy emitter, a RF cutting device, and a vibration device. The penetrating element may include a hollow needle and a guidewire arranged for advancement through tissue penetrated by the hollow needle. The penetrating element may include arrangement for penetrating between an aorta and a right-atrium.

The invention also provides an instrument for forming an aperture between cavities of two proximate body structures and deploying a connector in the aperture. The instrument includes a tubular structure arranged for placement in one of the cavities and having a sheath for deploying the connector, a tissue-cutting member arranged to form the aperture in tissue between the cavities, a guidewire following member, and a sheath arranged for deploying the connector in the aperture. The instrument may include a cut-tissue retention member. The instrument may further include a movement control member having an extracorporeal portion and arranged for moving the instrument along a guidewire, and the movement control member may include a radially expandable structure. The connector may include arrangement for traversing between lumens of an aorta and a right atrium. The tissue-cutting member may include a cutting aid selected from a group consisting of a thermal heating element, a laser energy emitter, a RF cutting device, and a vibration device. The guidewire following member may include arrangement for engaging a guidewire moved in a direction relative to the instrument. The instrument may include arrangement for endovascular use, and may be used in a beating heart.

The invention yet further provides an intra-luminal venous connector for fluid coupling a conduit placed in a cardiac vascular lumen to the vascular lumen. The connector includes an annularly enlargeable structure that, when annularly enlarged within a portion of a conduit arranged to carry arterial blood flow, couples the conduit with the vascular lumen. The structure includes arrangement for annular enlargement by a radially expandable structure placed within a portion of the elongated structure. When the structure is annularly enlarged and coupling the conduit with the vascular lumen, blood flow from the conduit into a right atrium is limited. The connector may include arrangement for endovascular implantation, and may be implanted in a beating heart.

The invention further provides an assembly for use in implanting an aorta-right atrium traversing connector. The assembly includes a guidewire path creation subassembly arranged for creating a guidewire pathway between an aorta and a right atrium, the subassembly including a first catheter having a distal tip arranged for placement into a cavity of a body structure and a lumen, a second catheter having a distal tip arranged for placement into a cavity of a body structure and a lumen, and a guidewire deployable from one catheter lumen and receivable by another catheter lumen and having a tissue penetrating element arranged to create a guidewire pathway by penetrating tissue between the lumens. The assembly further includes a guidewire guided instrument arranged for creating an aperture in response to the guidewire pathway between the aorta and the right atrium, and deploying a connector in the aperture. The guidewire-guided instrument may include a tubular structure arranged for endovascular placement, a sheath arranged for carrying and deploying the traversing connector, a tissue-cutting element, and a guidewire following member. The guidewire-guided instrument may include a movement control member for moving the instrument along a guidewire and having an extracorporeal portion. The assembly may further include a device arranged to provide arterial blood flow from the aorta to coronary venous system for venous retroperfusion of myocardium. The device includes an aorta-right atrium traversing connector arranged to receive arterial blood flow from the aorta, an arterial blood conduit in fluid communication with the traversing connector and a portion of the venous system, the conduit arranged for placement within the right atrium and the coronary sinus, and a venous connector that couples the conduit to the coronary venous system. The assembly may include arrangement for endovascular implantation, and may be implanted in a beating heart.

The invention provides a method of providing venous retroperfusion of myocardium. The method includes steps of acquiring arterial blood flow from an aorta, conveying the arterial blood flow through a right atrium, through a coronary sinus, and into a portion of a coronary venous system, and discharging the arterial blood flow in a portion of the coronary venous system for venous retroperfusion of a myocardium. The arterial blood flow may be acquired from the non-coronary aortic sinus. The step of acquiring the arterial blood flow may include the further step of directing the blood flow into an arterial blood conduit. The step of conveying the arterial blood flow may include the further step of routing an arterial blood conduit from acquisition in the aorta to a point of discharge in the coronary venous system. The step of providing the arterial blood flow may include the further step of coupling an arterial blood conduit with a lumen of the coronary venous system. The discharged arterial blood flow may include normal cardiac arterial blood flow phasing, and may include normal cardiac arterial blood pressure. The steps may be performed endovascularly, and may be performed in a beating heart.

The invention further provides a method of implanting a device that provides arterial blood flow from an aorta to a portion of a coronary venous system for venous retroperfusion of myocardium. The method includes the steps of placing an arterial catheter in the non-coronary aortic sinus at a position proximate to an aortic wall, placing a venous catheter in the right atrium at a position proximate to an atrium wall, and in approximate opposition to the arterial catheter, passing an arterial guidewire between the venous catheter and the arterial catheter, the guidewire passing through both the aortic wall and the atrium wall and having a proximal end, and placing a distal end of a venous guidewire into a lumen of the coronary venous system, the venous guidewire having a proximal end located adjacent to the proximal end of the arterial guidewire. The method also includes the steps of mounting portions of a lumen of the device moveably over the adjacent proximal ends of the venous guidewire and the arterial guidewire, a first portion being mounted on the arterial guidewire and the second portion being mounted on the venous guidewire, moving the mounted device along the guidewires into the right atrium, deploying the aorta-right atrium connector in the pathway and in fluid communication with the aorta, and deploying the venous connector in the selected portion of the venous system. The device may include an arterial blood flow conduit having a first portion with an aorta-right atrium traversing connector arranged to receive arterial blood from the aorta mounted on one end and second portion with a venous connector arranged to couple the conduit into a lumen of the coronary venous system mounted on a second end.

The invention additionally provides a device that provides venous retroperfusion of myocardium. The device includes means for acquiring an arterial blood flow from an aorta, means for conveying the acquired arterial blood flow through a right atrium and into a coronary sinus, and means for discharging the arterial blood flow into a portion of a coronary venous system.

The invention proves still another device that provides arterial blood flow from the aorta to a vascular structure for perfusion of cardiac tissue. The device includes a connector arranged to receive arterial blood flow from the aorta, an arterial blood conduit in fluid communication with the connector and the vascular structure, the conduit arranged for placement within a heart chamber and the vascular structure, and a connector arranged to couple the conduit with the vascular structure. The vascular structure may be a vein or an artery. The device may be arranged for endovascular implantation in a beating heart.

These and various other features as well as advantages that characterize the present invention will be apparent from a reading of the following detailed description and a review of the associated drawings.

### BRIEF DESCRIPTION OF THE DRAWINGS

The features of the present invention which are believed to be novel are set forth with particularity in the appended claims. The invention, together with further objects and advantages thereof, may best be understood by making reference to the following description taken in conjunction with the accompanying drawings, in the several figures of which like referenced numerals identify like elements, and wherein:

FIG. 1 depicts a human heart from above without the non-coronary vascular structures;

FIG. 2 depicts a myocardium of a human heart including a lattice of capillaries that drain deoxygenated blood into intramyocardial veins and the Thebesian system;

FIG. 3 illustrates the heart of FIG. 1 after implantation of a device providing arterial blood flow for venous retroperfusion of ischemic myocardium, in accordance with the invention;

FIG. 4 is a front view of a patient illustrating a guidewire pathway created between the aorta and the right atrium, the guidewires are in a position for implantation of the device providing arterial blood flow, and percutaneous endovascular introduction sites, in accordance with the invention;

FIG. 5 is a view similar to FIG. 1 and illustrates distal tips of a venous side catheter and an arterial side catheter in position for a guidewire to create a guidewire pathway between the right atrium and the non-coronary aortic sinus portion of the aorta, in accordance with the invention

FIG. 6 is a view similar to FIG. 1 and illustrates final steps of creating the guidewire pathway using a guidewire, in accordance with the invention;

FIG. 7 is a view similar to FIG. 1 and illustrates placement of the guidewires in preparation for placing the device providing arterial blood flow in the heart, in accordance with the invention;

FIG. 8 is a view similar to FIG. 1 and illustrates the arterial blood flow conduit slideably carried on the guidewires and placed in the heart in preparation for implantation, in accordance with the invention;

FIG. 9 is a cross-sectional perspective view illustrating a venous connector in an initial configuration partially mounted on the distal end of an arterial blood flow conduit and carried on a partially expanded balloon catheter, in accordance with the invention;

FIG. 10 is similar to FIG. 9, and illustrates a venous connector in a fully expanded configuration engaging the distal end of the arterial blood flow conduit with the vascular lumen of the great cardiac vein, in accordance with the invention;

FIG. 11 is similar to FIG. 9, and illustrates a configuration where the balloon catheter has been deflated to an unexpanded configuration for removal from the patient, in accordance with the invention;

FIG. 12 is a cross-sectional perspective view illustrating an initial step for cutting an aperture through tissue between cavities of two body structures employing an assembly moveably carried on a guidewire, in accordance with the invention;

FIG. 13 is similar to FIG. 12, and illustrates intermediate steps in cutting an aperture through tissue between cavities of the right atrium and the aorta, and an initial step in deploying the traversing connector, in accordance with the invention;

FIG. 14 is similar to FIG. 12, and illustrates another intermediate step in cutting an aperture through tissue between the right atrium and the aorta, and another step in deploying the traversing connector, in accordance with the invention;

FIG. 15 is similar to FIG. 12, and illustrates a final configuration of the traversing connector implanted in apertures created between the right atrium and the non-coronary aortic sinus, in accordance with the invention;

FIG. 16 illustrates an assembly employing a knot pusher for sealing a sealable exit opening of an arterial blood flow conduit, in accordance with the invention;

FIG. 17 is a perspective view illustrating the inlet member in a compressed and pre-deployment configuration, in accordance with the invention;

FIG. 18 is similar to FIG. 17, and illustrates the inlet member in an expanded and deployed configuration, with engaging elements radially extended, in accordance with the invention;

FIG. 19 is a perspective view illustrating the positioning member in a compressed and pre-deployment configuration, in accordance with the invention; and

FIG. 20 is similar to FIG. 19, and illustrates the positioning member in an expanded and deployed configuration with engaging elements and braces radially extended, in accordance with the invention.

### DETAILED DESCRIPTION

In the following detailed description of exemplary embodiments of the invention, reference is made to the accompanying drawings, which form a part hereof. The detailed description and the drawings illustrate specific exemplary embodiments by which the invention may be practiced. These embodiments are described in sufficient detail to enable those skilled in the art to practice the invention. It is understood that other embodiments may be utilized, and other changes may be made, without departing from the spirit or scope of the present invention. The following detailed description is therefore not to be taken in a limiting sense, and the scope of the present invention is defined only by the appended claims.

Throughout the specification and claims, the following terms take the meanings explicitly associated herein unless the context dictates otherwise. The meaning of “a”, “an”, and “the” include plural references. The meaning of “in” includes “in” and “on.” Referring to the drawings, like numbers indicate like parts throughout the views. Additionally, a reference to the singular includes a reference to the plural unless otherwise stated or inconsistent with the disclosure herein.

Briefly stated, aspects of the present invention generally relate to methods and apparatus for use in endovascular and intraoperative procedures providing arterial blood flow for perfusion of ischemic myocardium. Aspects of the present invention provide a conduit between a non-coronary sinus of the aorta and a coronary vein, the conduit traversing a portion of the right atrium. The conduit is located entirely within the heart and aorta. Arterial blood flows from the aorta through the conduit and into the coronary venous circulation towards the ischemic region of the heart. All procedures described herein may be performed endovascularly, and further may be performed while the patient's heart is beating.

The description of the present invention is organized as follows: First, the anatomy of a heart, and its arterial and coronary vascular systems relevant to the present invention are described. Next, a heart illustratively treated with methods of and apparatus in accordance with the present invention is described. This is followed by a description of a method for placing an apparatus of the present invention within the heart, including several components of various embodiments of the apparatus of the present invention. Finally, additional details are illustrated of several components of various embodiments of the invention.

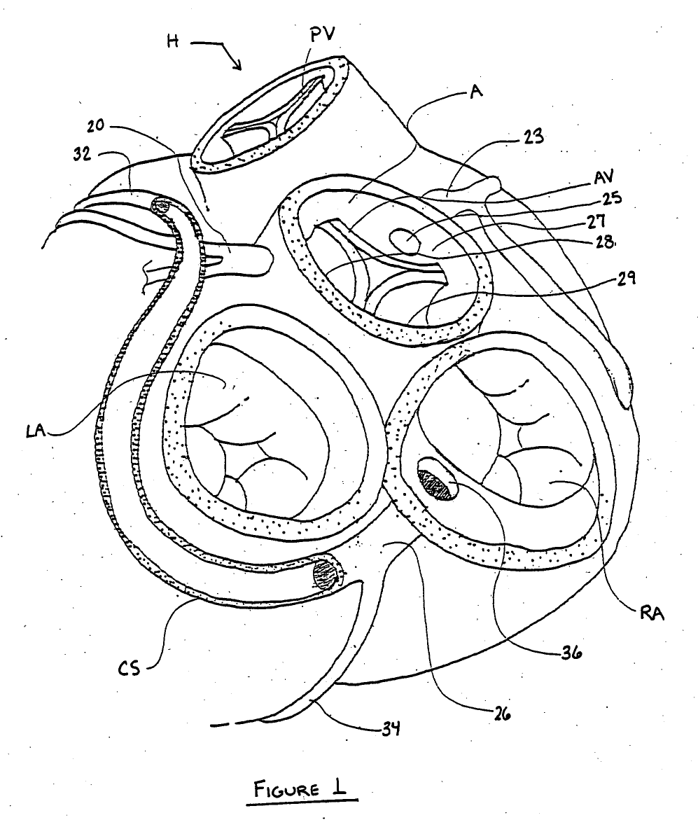

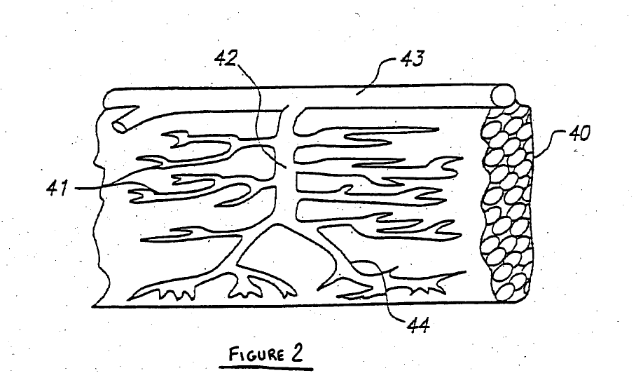

FIGS. 1 and 2 describe various features of the human. heart relevant to the present invention. FIG. 1 depicts a human heart H from above without the non-coronary vascular structures. The illustration includes the aortic valve AV, the pulmonary valve PV, the right atrium RA, and the left atrium LA. The coronary arterial system comprises a left coronary artery 20 and a right coronary artery 23, which branch into sub-branches supplying the heart with oxygenated blood. Both coronary arteries receive blood flow from openings in the coronary sinuses, the right coronary artery 23 being supplied by opening 25 in the coronary sinus formed with the right semilunar cusp 27 of the aortic valve AV. The left coronary artery 20 is supplied by an opening (not shown) in the coronary sinus formed with the left semilunar cusp 28 of the aortic valve AV. The non-coronary sinus is formed with the posterior semilunar cusp 29 (hereafter called the non-coronary aortic sinus 29), and does not have an arterial opening. The non-coronary aortic sinus 29 is located relatively close to the right atrium RA, the walls of both structures being nearly in contact. The blood flow to the coronary arteries 20 and 23 at the level of the coronary aortic sinuses 27 and 28 occurs during diastole.

The heart H receives deoxygenated blood from the venous system into right atrium RA. The coronary sinus CS discharges deoxygenated blood flowing in the coronary venous system through the coronary ostium 36 and into the right atrium RA. The coronary sinus CS provides drainage for great cardiac vein 32, middle cardiac vein 34, and other veins that are not shown. The cardiac venous system further includes cardiac veins that drain directly into the right atrium RA as described in FIG. 2.

With respect to FIG. 2, myocardium 40 includes a lattice of capillaries 41 that drain deoxygenated blood into intramyocardial veins 42. From intramyocardial veins 42, a fraction of the blood drains into the cardiac veins via subepicardial veins 43, while the remainder drains through the Thebesian veins 44 directly into the atrial and ventricular cavities. It has been reported in healthy human hearts that approximately 70% of the deoxygenated blood is drained through the coronary sinus CS, while the remaining 30% is drained into the heart via the lymphatic system and the Thebesian veins 44. It has likewise been reported that when individual components of the venous system (i.e., the coronary sinus, lymphatic system and Thebesian veins) are occluded, the flow redistributes itself through the remaining unoccluded channels.

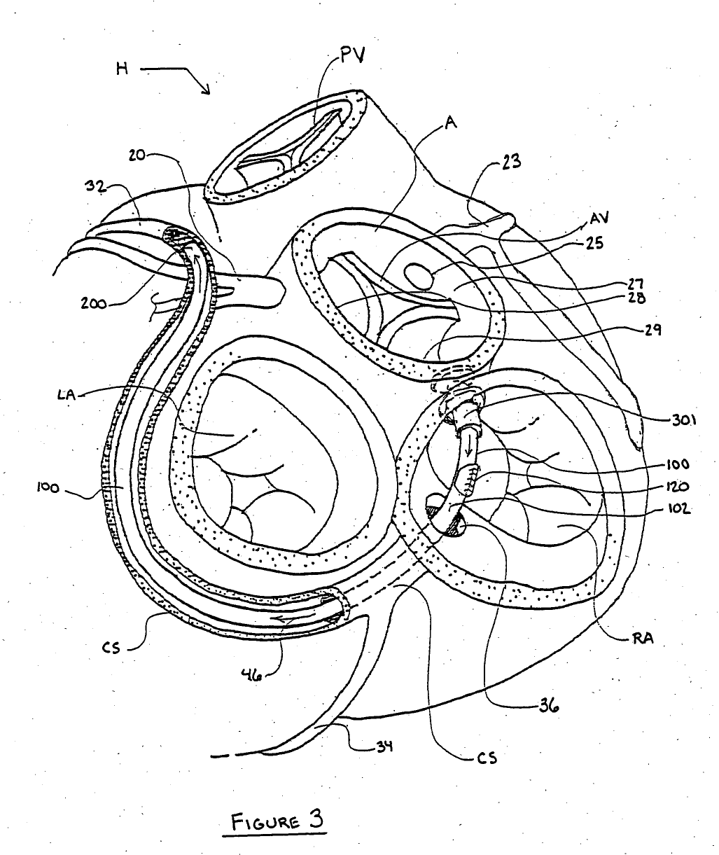

In FIG. 3, the heart H of FIG. 1 is shown after implantation of a device providing arterial blood flow 100 for venous retroperfusion of ischemic myocardium, in accordance with the invention. The device 100 includes an arterial blood flow conduit 102, a venous connector 200, and a traversing connector 300. The conduit 102 has been routed through the right atrium RA and the coronary sinus CS, terminating in the great cardiac vein 32. The traversing connector 300 acquires arterial blood flow 46 from the non-coronary aortic sinus 29 of the aorta A and provides it to the conduit 100. Venous connector 200 couples the blood flow 46 of conduit 102 into the lumen of great cardiac vein 32 for venous retroperfusion of myocardium, and limits the blood flow from flowing toward the right atrium RA.

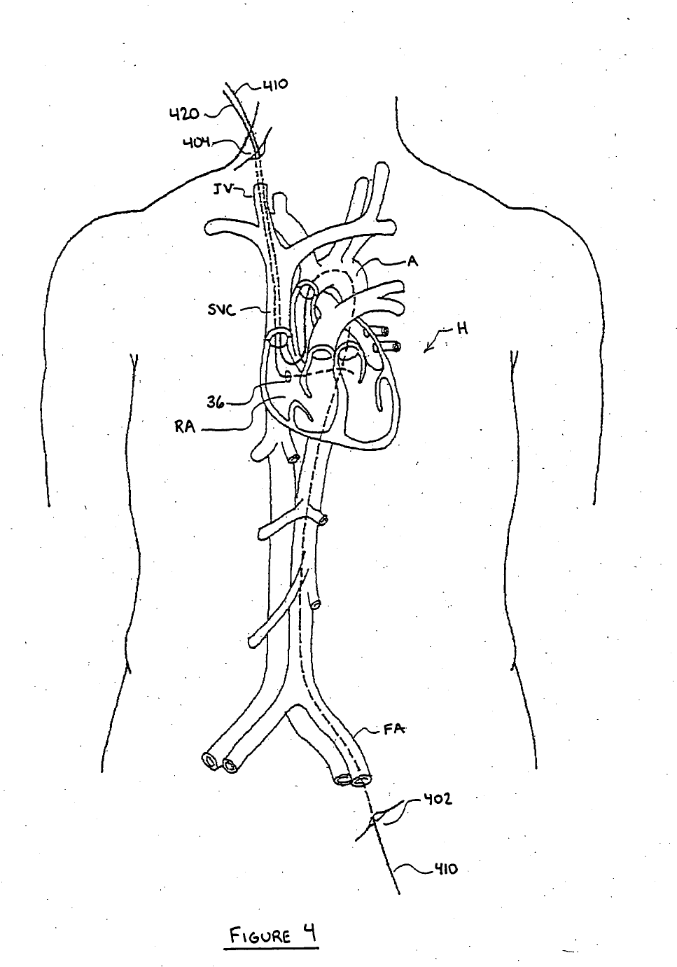

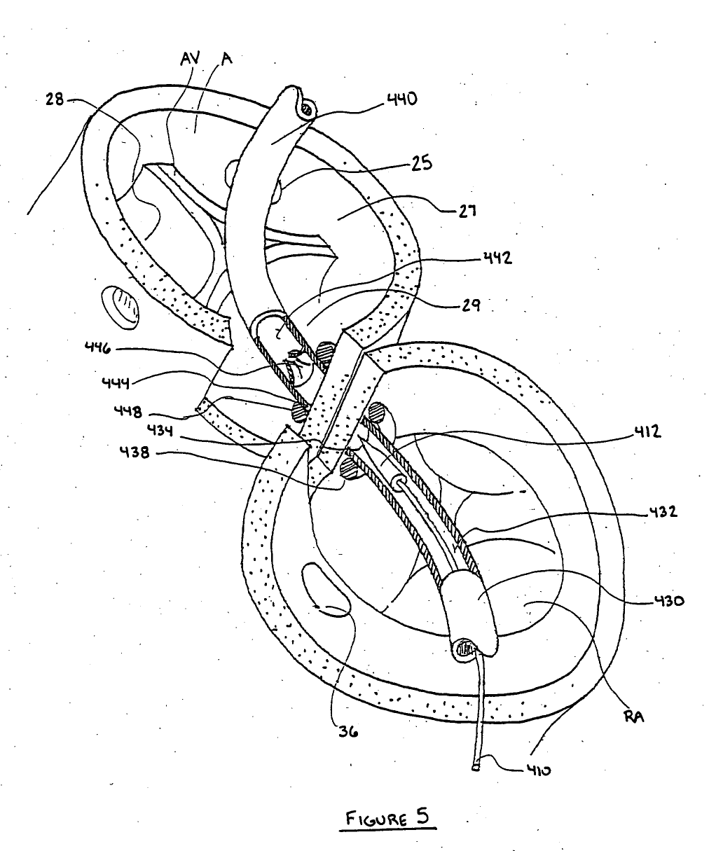

FIGS. 4-7 illustrate steps employing endovascular methods for placing a guidewire 410 between aorta A and right atrium RA, and a guidewire 420 into the coronary sinus CS and the great cardiac vein 32 of the coronary vascular system, in accordance with the invention. FIGS. 5-7 are views similar to FIG. 1.

Various imaging modalities may be used to aid in accomplishing the positioning of the various apparatus and devices described herein, such as fluoroscopy with angiography or ultrasound (intravascular or intracardiac) or a combination of the two. Alternatively, other imaging technologies may be used. The devices and apparatus may include substances that enhance imaging. While preparation for and implantation of the device 100 is described herein by endovascular methods, device 100 may be implanted by another method or procedure, including an open surgical setting or other interventional cardiology setting.

FIG. 4 is a front view of a patient illustrating the heart H and vascular system after creation of a guidewire pathway between the aorta A and the right atrium RA, and placing the guidewires 410 and 420 in a position for implantation of device 100. FIG. 4 also illustrates using femoral artery FA for an arterial percutaneous endovascular introduction site 402 and a jugular vein JV for a venous endovascular introduction site 404. Possible other arterial introduction sites include the radial artery or aorta. Possible other venous introduction sites include subclavian vein, femoral vein, or superior vena cava SVC.

FIG. 5 is a view similar to FIG. 1 illustrating distal tips of a venous side catheter 430 and an arterial side catheter 440 in position for a guidewire 410 to create a guidewire pathway between the right atrium RA and the non-coronary aortic sinus 29 portion of aorta A, in accordance with the invention. The catheters 430 and 440 are arranged for endovascular use and are preferably steerable. Each catheter has a lumen, 432 and 442, respectively, for passage of guidewires, and a distal tip, 434 and 444, respectively. The catheters may be made of any material suitable for endovascular cardiac procedures. The distal tips 434 and 444 are arranged for alignment of the distal portions of lumens 432 and 434 in vivo, such that a guidewire deployed from one lumen can be received in the other lumen. In an embodiment illustrated in FIG. 5, catheter 430 is arranged to deploy the guidewire 410, and catheter 440 is arranged to receive it. Catheter 440, includes a catching member 446 arranged to engage guidewire 410 when it enters lumen 442. Catching member 446 may be any mechanism or device arranged to engage either the guidewire 410 or the penetrating element 412 and preclude movement of the guidewire 410 other than in the direction of advancement 414. In an alternative embodiment, the catching member 446 may be a lasso mechanism arranged to snare the guidewire 410 after it passes through the aortic and atrial tissue layers. Catheters 430 and 440 may each be arranged for the specific vascular or cardiac structures into which they are intended for placement. For example, the distal tip 434 of catheter 430 may be formed to aid in placing it proximate to a preselected portion of the right atrium wall. Likewise, the distal tip 444 of arterial catheter 440 may be formed to aid in placing it in the non-coronary aortic sinus 29 and against the aortic wall.

The distal tips 434 and 444 may carry alignment devices 438 and 448, respectively. Alignment devices 438 and 448 may be any device or combination of devices suitable for in vivo alignment of the distal portions of the lumens 432 and 434, such that a guidewire deployed from one lumen can be received in the other lumen. Alignment devices 438 and 448 are illustrated in FIG. 5 as magnets 438 and 448 carried on distal tips 434 and 444. The polarization of magnets 438 and 448 is arranged for self-alignment of the distal portions of the lumens 432 and 434. The magnets 438 and 448 can have any shape suitable for the intended use, such as a donut shape. The magnets are arranged to attract and align with each other in only one configuration, such that when the guidewire 410 with its penetrating element 412 is deployed from the lumen 432 of catheter 430 it is receivable by the lumen 442 of catheter 440. In alternative embodiments, one alignment device may be an electrical signal source and the other an electrical signal sensor; one alignment device may be an ultrasound source and the other an ultrasound sensor; or one alignment device may be a light source and the other a light sensor. In these alternative embodiments, the source and the sensor are used to guide the distal tips 434 and 444 into proximity. The distal tips 434 and 444 may include a substance viewable with an imaging device. In a further alternative embodiment, one or both lumens 432 and 442 may be usable for ejecting a substance viewable with an imaging device, or an additional lumen may be provided in one or both catheters for ejecting a viewable substance. The ejected viewable substance may be used to guide the distal tips 434 and 444.

Guidewire 410 includes a penetrating element 412 arranged to penetrate tissue between distal tips 434 and 444, and which may be further arranged to engage catching member 446. Guidewire 410 may be any size, shape, and configuration suitable for use in vascular procedures. In an embodiment, guidewire 410 is approximately 0.014 inches in diameter. The penetrating element 412 may be a sharpened distal end of guidewire 410, or may be an element carried preferably on the distal end of guidewlre 410. Penetrating element 412 may include a device to aid penetration, such as a thermal heating element, a laser energy emitter, a RF cutting device, or a vibration device. In an alternative embodiment, the penetrating element 412 may include a hollow needle deployed from a distal tip and a guidewire arranged for advancement through tissue penetrated by the hollow needle.

FIG. 5 also illustrates initial steps in percutaneous endovascular implantation of device 100. A step includes introducing the distal tip 444 of arterial catheter 440 at site 404, and the distal tip 434 of the venous catheter 430 at site 404. These sites are illustrated in FIG. 3. The distal tip 444 is steered into the aorta A to a position at a level of the non-coronary sinus 29 and proximate to an aortic wall. The distal tip 434 is steered into the right atrium RA to a position adjacent to the non-coronary sinus 29. Steering may be by any method, including visualization methods. After the above step, the distal tips 434 and 444 are in proximity to each other, separated by the tissues of the aortic wall and the right atrium wall.

Another initial step includes aligning the distal portions of the lumens 432 and 442. Once in proximity to each other, the magnets 438 and 448 carried on the distal tips will attract and align with each other, cause the distal tips 434 and 444 to contact the walls, and align the distal portions of lumens 432 and 442, such that a guidewire deployed from one lumen can be received in the other lumen. If an alternative embodiment is used where the alignment is aided by a signal source, the source, preferably carried in the distal tip 444 of arterial catheter 440, is activated and the distal tip of the other catheter, is maneuvered until a maximum signal is sensed by the sensor, indicating alignment. If a light source is used, the source is also preferably carried in the distal tip 444 of arterial catheter 440. The sensor may be an optical lens or photo sensor carried on the other distal tip, which is maneuvered until a maximum light is received, indicating alignment.

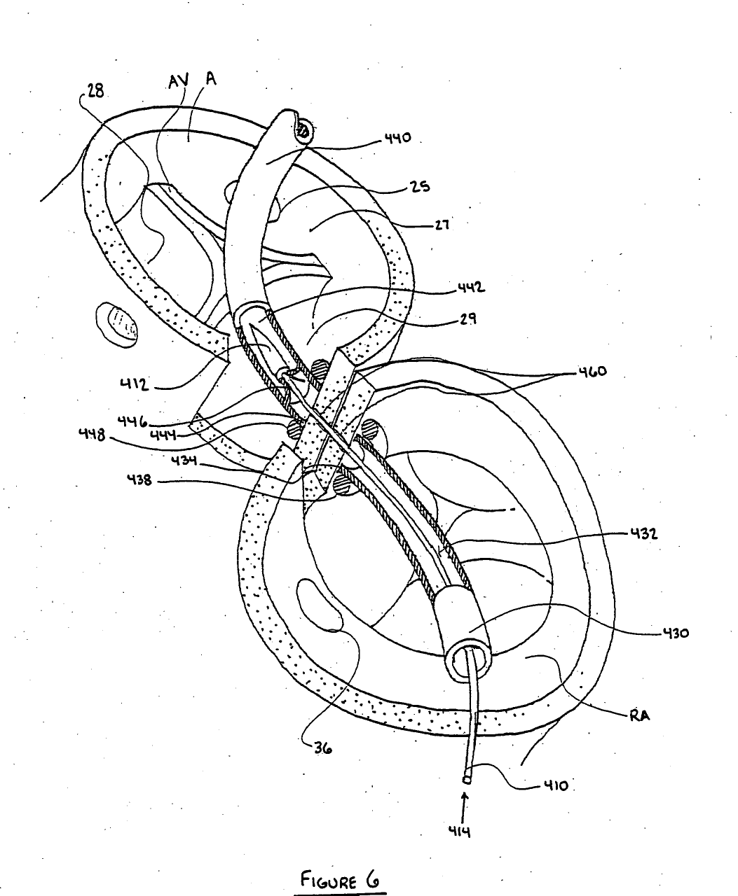

FIG. 6 illustrates final steps of creating the guidewire pathway 460 using guidewire 410. The guidewire 410 can be introduced into the heart H using either the venous catheter 430 at site 404 or the arterial catheter 440 at site 402. FIG. 6 illustrates introducing the guidewire 410 using the venous catheter 430. A step includes advancing the guidewire 410 and its penetrating element 412 through the lumen 432 and into proximity with a tissue wall of the right atrium RA. Another step includes further advancing the guidewire 410 to deploy the penetrating element 412 from a lumen 432 and to penetrate through the right atrial wall and the aortic wall tissue between the deploying lumen 432 and the receiving lumen 442. If the penetrating element 412 includes a device to aid penetration, the device is activated. If the penetrating element 412 includes a hollow needle, the guidewire 410 may be advanced after penetration by the hollow needle. The needle can be retracted after the guidewire 410 is advanced into the receiving catheter. The guidewire 410 and its penetrating elements 412 are small enough to minimize bleeding when penetrating tissue, and the penetration is anticipated to be self-healing.

As used in these specifications, “guidewire pathway” means any guiding path or pathway between the right atrium RA and the non-coronary aortic sinus 29, and typically will have sufficient diameter for passage of a guiding device, such as a guidewlre. A “guidewire pathway” may include any kind of guiding path arranged to guide movement of any-device between the right atrium RA and the non-coronary aortic sinus 29.

In a further step, the guidewire 410 is advanced into the lumen 442 of the arterial catheter 440. If the receiving catheter 440 includes a catching member 446, the guidewire 410 is advanced until the catching member 446 or the penetrating element 412 engages it. Guidewire 410 is further advanced until a portion of the guidewire 410 and the penetrating element 412 is exteriorized as illustrated in FIG. 4. At this point, the guidewire 410 extends from outside the body at site 402 into the arterial catheter 430, through the aortic wall and the right atrial wall, into the venous catheter 440, and outside the body again at site 404. Alternatively, instead of advancing the guidewire 410 to exteriorize it, after the guidewire 410 engages the catching member 446, the receiving catheter 440 may then be withdrawn from the patient. This will exteriorize the guidewire 410.

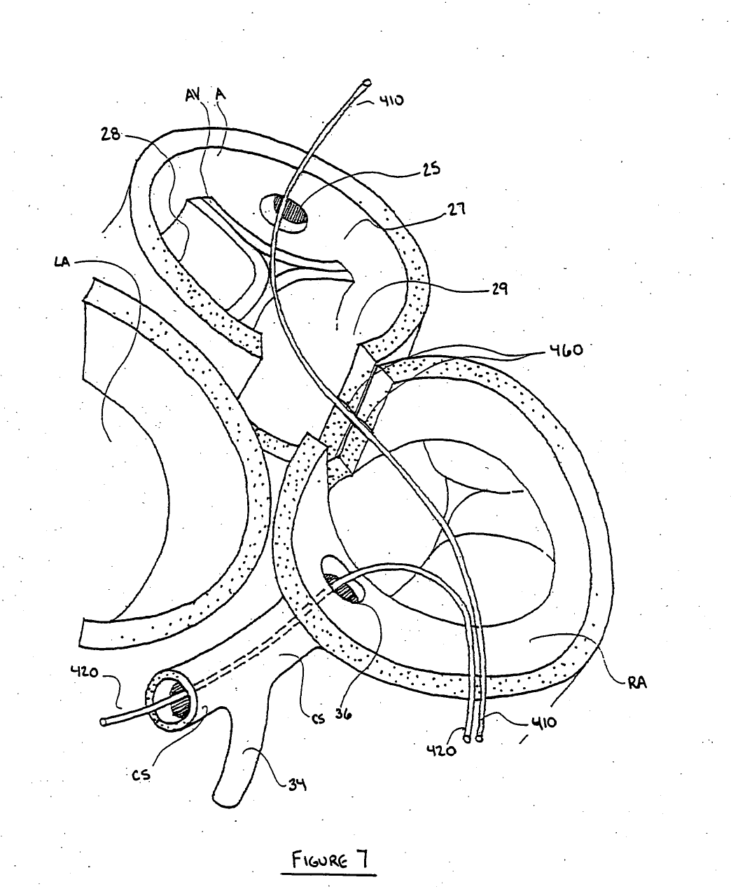

FIG. 7 is a view similar to FIG. 1, and illustrates placement of the guidewires 410 and 420 in preparation for placing the device 100 in the heart H. FIG. 7 illustrates guidewire 410 placed in guidewire pathway 460 as described above. A step includes withdrawing both catheters 430 and 440 from the patient.

Guidewire 420 may be any size, shape, and configuration suitable for use in vascular procedures. In an embodiment, guidewire 420 is approximately 0.035 inches in diameter.

A step in placing the guidewire 420 includes introducing a coronary venous guiding catheter (not shown) at site 404 of FIG. 4, and advancing the catheter to the coronary sinus ostium 36 in the right atrium RA. The coronary venous guiding catheter is further advanced into the coronary sinus CS, and to a position that is proximate to a selected location in the coronary venous system for discharging the arterial blood flow 46. Once the coronary venous catheter is in position, the guidewire 420 is advanced in a lumen of the coronary venous catheter until its distal end (not shown) is placed in the selected location, or preferably slightly distal thereof. As another step, the coronary venous catheter is then removed from the patient leaving the guidewire 420.

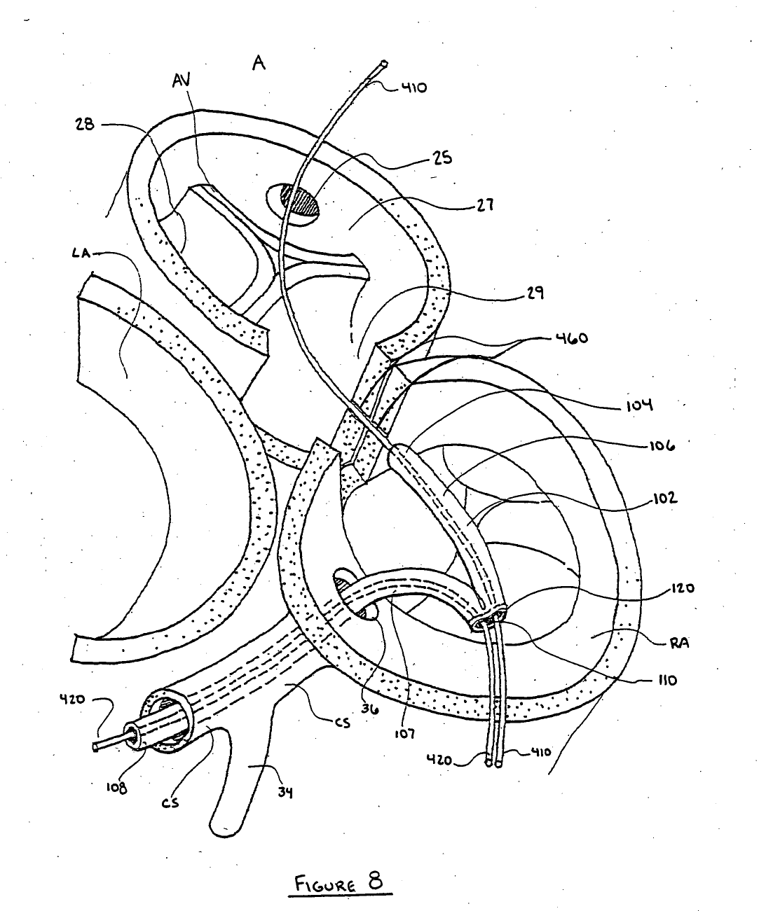

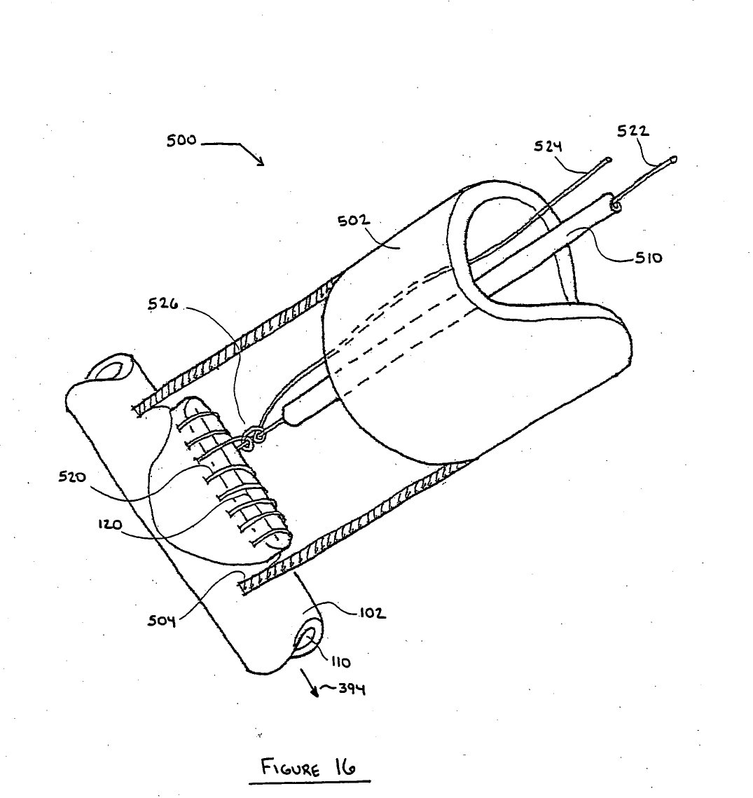

FIG. 8 is a view similar to FIG. 1 and illustrates a step where the arterial blood flow conduit 102 of device 100 is slideably carried on the guidewires 410 and 420 and placed in the heart H in preparation for implantation, in accordance with the invention. The venous connector 200 and the traversing connector 300 are omitted from FIG. 8 for clarity. The arterial blood flow conduit 102 of device 100 comprises a tubular member having a first end 104 adapted to be coupled to the aorta-right atrium traversing connector 300 (not shown), a second end 108 adapted to be coupled to the venous connector 200 (not shown), an intermediate portion located between the ends 104 and 108 and including a first region 106 near the first end 104 adapted to be placed in the right atrium RA and a second region 107 near the second end 108 adapted to be placed into the coronary ostium 36, through the coronary sinus CS and into a portion of the venous system. The arterial blood flow conduit 102 also comprises a lumen 110 arranged to provide fluid communication between the ends 104 and 108, and comprising a flexible material. The intermediate portion of conduit 102 includes a sealable exit opening 120 allowing passage over guidewires 410 and 420. The sealable exit opening 120 may be arranged for sealing against blood leakage by any method known to those in the art, including a purse string suture as illustrated in FIG. 16, or a self-sealing diaphragm, such as used for introducer sheets in interventional procedures. The arterial blood conduit 102 may be formed from an autologous vein or artery, or a non-autologous or a synthetic material. Possible autologous veins include a saphenous vein. Possible synthetic materials include any biocompatible material known to those in the art, including polyvinyl chloride, polyethylene, polytetrafluoroethylene (PTFE), and ePTFE. The conduit 102 will be approximately 8 cm long, depending on the selected location for placement of the venous connector 200, and will have an inside diameter of approximately 3 mm.

The device 100 is placed within the right atrium RA in preparation for implantation. An initial step includes placing portions of the lumen 110 of the device 100 slideably over adjacent extracorporeal portions of the guidewires 410 and 420 at site 404. The extracorporeal portion of the guidewire 410 is placed in the lumen 110 of the first end 104 with the aorta-right atrium traversing connector 300 (not shown) mounted, and the extracorporeal portion of the guidewire 420 is placed in the lumen 110 of the second end 108 with the venous connector 200 (not shown) mounted. As the device 100 and the ends 104 and 108 are initially advanced, the extracorporeal portions of the guidewires 410 and 420 both pass out of the lumen 110 at a sealable exit opening 120 and remain extracorporeal. The ends 104 and 108 of the device 100 are advanced over the guidewires 410 and 420 into. the jugular vein at site 404, into the superior vena cava SVC, and toward the right atrium RA of the heart H. The ends 104 and 108 are advanced using any pushing apparatus known to those in the art, such as two balloon catheters with the expandable portions partially inflated near the distal ends (104, 108) of the conduit 102 to engage it. Alternatively, the pushing apparatus may be a small caliber tubular structure of a given stiffness or with a hollow center that allows stylets of different stiffness to be introduced. The device 100 is advanced into the right atrium RA and the coronary sinus CS until it is placed approximately as illustrated in FIG. 8.

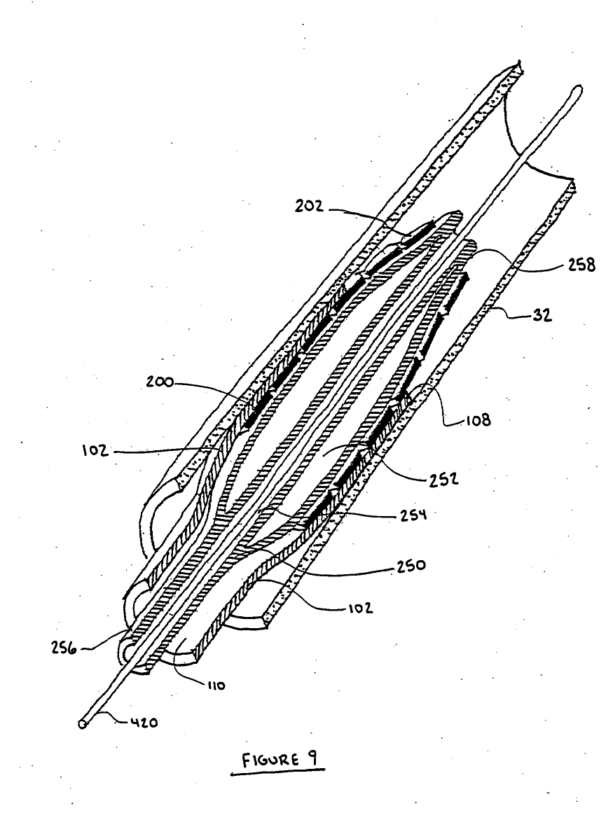

FIGS. 9-11 are cross-sectional perspective views illustrating the connector 200 and the second distal end 108 of conduit 102 carried on a balloon catheter 250 and moveable along the guidewire 420 for placement in the great cardiac vein 32, in accordance with the invention. FIG. 9 is a cross-sectional perspective view illustrating the connector 200 in an initial configuration, partially mounted on the distal end 108 of conduit 102 and carried on the balloon catheter 250, which is in a partially expanded configuration.

Venous connector 200 is a balloon expandable structure, such as a stent, and its distal end may include a tapered tip portion 202 arranged to facilitate advancement into the venous system. The venous connector 200 may have the configuration of a conventional vascular stent with added features to ensure the connector is partially in contact with the inside of the vein and creates a partial or complete seal with the vein. The connector 200 may be laser cut Nitinol or stainless steel tube expanded into a mesh-like structure. The connector 200 may include members to facilitate engagement between the connector, the conduit 102, and the venous system, such as barbs.

The balloon catheter 250 includes a lumen 254 arranged for following a guidewire, an expansion member 252, and an elongated shaft 256 having an extracorporeal portion arranged for advancing and retracting the balloon catheter 250. The balloon catheter 250 may be any type of expandable catheter suitable for endovascular use, and those having a relatively short length and larger diameter may be particularly suited for use in accordance with the invention. The catheter 250 and the connector both may have tapered distal ends (202, 258), which may facilitate advancement through the venous structures and the heart H.

Prior to insertion into the venous structure used to access the right atrium RA, the distal end 108 of the conduit 102 is placed over an outside periphery of the unexpanded connector 200 covering approximately one-half of its length as generally illustrated in FIG. 9. Another step prior to insertion includes placing the expansion member 252 of the balloon catheter 250 into the sealable exit opening 120 and advancing it toward the distal end 108 until positioned within unexpanded connector 200, such that expanding the expansion member 252 will expand the connector 200. The expansion member 252 is then partially expanded to engage connector 200 and to annularly enlarge connector 200 sufficient to engage a portion of the conduit 102 proximate to the distal end 108, and forming a connector assembly 260.

Another step includes placing the extracorporeal end of guidewire 420 inside the lumen 254 of the balloon catheter 250 at its tapered distal end 258, thus slideably engaging the guidewire 420. The connector assembly 260 is advanced along guidewire 420 into the coronary sinus CS and the great cardiac vein 32 as described in conjunction with FIG. 8. The tapered end 258 of the balloon catheter 250 is advanced along guidewire 420 to a preselected location in the great cardiac vein 32 for discharge of arterial blood flow from the device 100 for venous retroperfusion of ischemic myocardium of the heart H. The progress and position of the tapered end 258 of the balloon catheter 250 may be monitored by X-ray fluoroscopy.

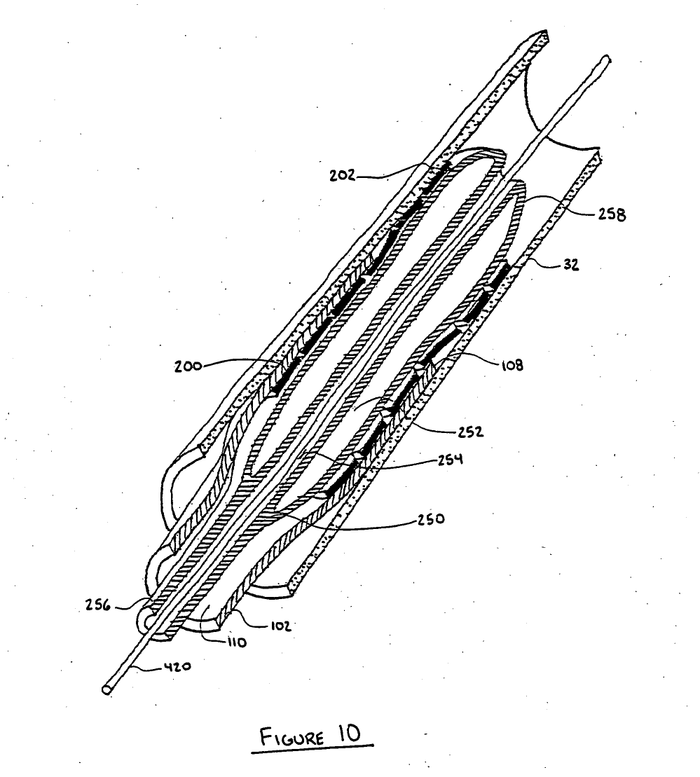

Once the distal end 108 is at the preselected location in the great cardiac vein 32, another step involves fully expanding the balloon catheter 250. When the balloon catheter 250 is in a fully expanded configuration, the connector 200 is annularly enlarged within the conduit 102 and engages the distal end 108 of the conduit 102 with the vascular lumen of the great cardiac vein 32. The annularly expanded connector 200 also directly engages the vascular lumen of the great cardiac vein 32. FIG. 10 is a cross-sectional perspective view illustrating this intermediate step. The vascular lumen of a cardiac vein has a normal diameter of about 4 to 4.5 mm when under typical venous pressure, and might expand further in response to expansion of the connector 200. The connector 200 may be annularly enlarged to a diameter greater than the normal vascular lumen diameter to aid engagement between the conduit 102, the connector 200, and the vascular lumen of the great cardiac vein 32.

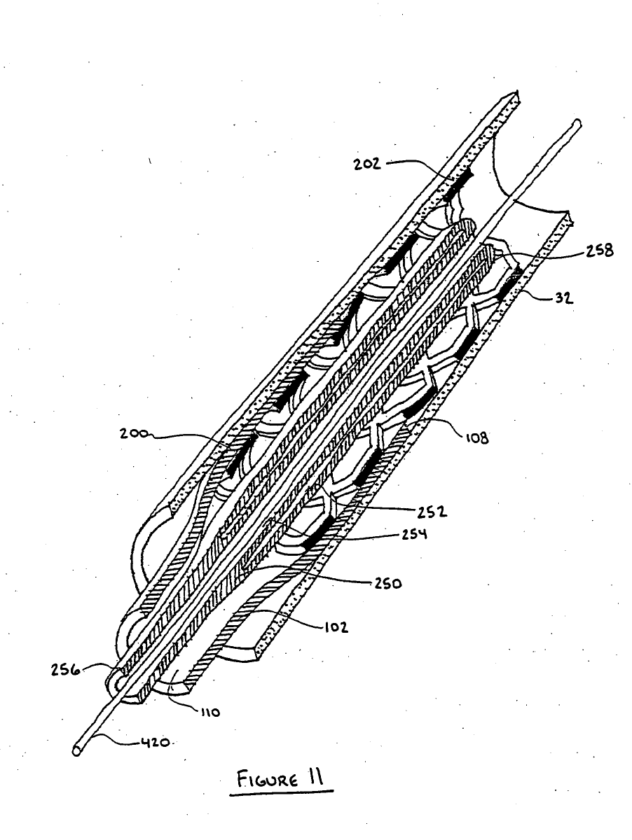

FIG. 11 is similar to FIG. 9, and illustrates a configuration where the balloon catheter 250 has been deflated to an unexpanded configuration. Once the connector 200 has been annularly enlarged and is directly engaging the vascular lumen, and is further engaging the conduit 102 with the vascular lumen, a final step includes deflating the balloon catheter 250 to an unexpanded configuration for removal. This unexpanded configuration leaves the connector 200 in place and engaging the conduit 102 with the vascular lumen, thus fluid coupling the conduit 102 to the vascular lumen. The engagement may be confirmed by visualization methods. Another final step includes withdrawal of the balloon catheter 250 and the guidewire 420 out of the conduit 102 at sealable exit opening 120 and from the patient. The fluid coupling of conduit 102 to the wall of the vein 32 forms a fluid tight seal directing aortic blood flow from conduit 102 into the vein 32. Retrograde flow through the conduit 102 will be largely or completely directed toward the venous microcirculation instead of the right atrium RA.

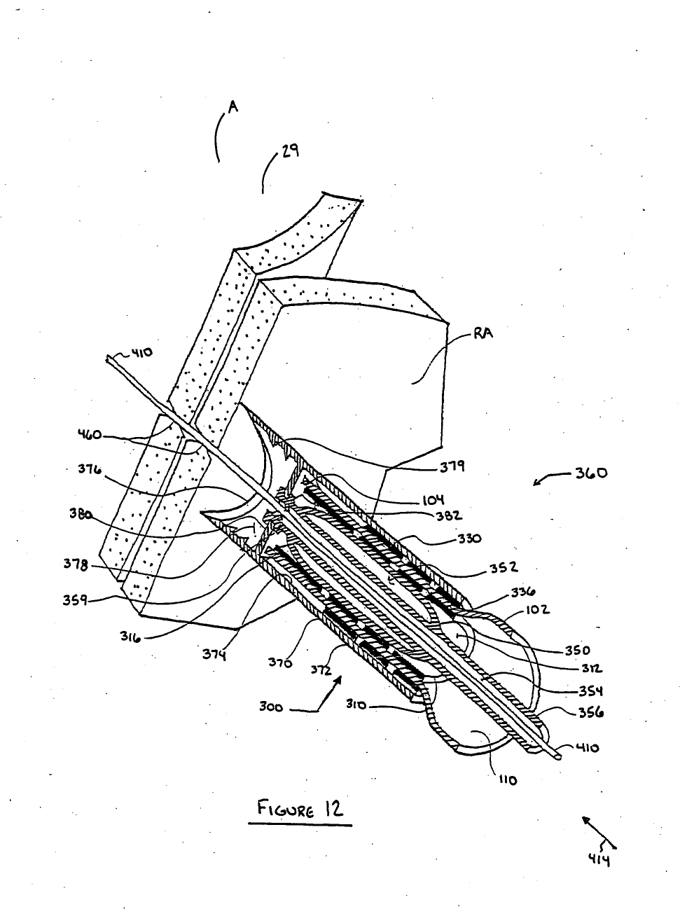

FIGS. 12-15 are cross-sectional perspective views illustrating employing an assembly 360 for cutting an aperture through tissue between cavities of two body structures and deploying a traversing connector 300 in the aperture, in accordance with the invention. In FIGS. 12-15, aspects of the invention are illustrated cutting an aperture between a right atrium and an aorta, and deploying the traversing connector 300 using endovascular methods.

FIG. 12 is a cross-sectional perspective view illustrating the assembly 360 moveably carried on a guidewire 410 and located proximate to a portion of the right atrium interior wall. The guidewire 410 passes through the guidewire pathway 460 that is proximate to the non-coronary aortic sinus 29, and both ends of which are outside of the patient's body in an arrangement similar to FIGS. 4, and 9-11. Assembly 360 includes a tissue cutter and deployment instrument illustrated as a cutter/deployer 370, a traversing connector 300 in a collapsed configuration, and a balloon catheter 350, all arranged for endovascular procedures in a beating heart.

The tissue cutter/deployer 370 includes a tubular structure 372, a sheath 374, a tissue-cutting member 376, a cut-tissue retention member 378, a guidewire following member 380, and a guidewire engaging member 382. While illustrated as a round elongated structure, the tubular structure 372 may have any shape suitable for its intended use, and typically may be round with an outside diameter of between approximately 4 to 4.5 mm, and may be made from any suitable material, such as stainless steel. The tissue-cutting member 376 has a sharpened circumferential edge arranged to cut an aperture when advanced through tissue, and typically will be formed on the tubular structure 372. While illustrated as formed on a perpendicular cross-sectional plane, the cutting member 376 may be formed on any plane, may have a pointed portion to make initial contact with a small portion of tissue, and may have an irregular edge. Further, the cutting member 376 may be a separate apparatus carried on the tubular structure 372. The cutting member 376 may include a device to aid cutting, such as a thermal heating element, a laser energy emitter, a RF cutting device, or a vibration device. The cut-tissue retention member 378 retains for removal the cut tissue 390, and prevents the cut tissue 390 from being released into the patient. The tissue retention member 378 may be a chamber in the tubular structure 372 proximate to the cutting member 376, and retention of the cut tissue 390 may be assisted by one or more other members, such as barbs 379.

The guidewire following member 380 may be any structure allowing the cutter/deployer 370 to follow a guidewire, and is illustrated as a portion of the tubular structure 372 having an opening dimensioned for following a guidewire. The guidewire engaging member 382 is arranged for engaging a guidewire moved in a direction relative to the cutter/deployer 370. The engaging member 382 may be a pawl that frictionally engages the guidewire. The guidewire engaging member 382 is illustrated in FIGS. 12-14 incorporated into the balloon catheter 350. The sheath 374 is arranged to carry the traversing connector 300 in a collapsed configuration for endovascular delivery into an aperture cut by the cutting member 376, and for deployment therein. The sheath 374 may be an interior cavity of cutter/deployer 370 having a periphery arranged to carry the traversing connector 300, and further arranged to allow deployment by a method compatible with the configuration of the traversing connector 300.

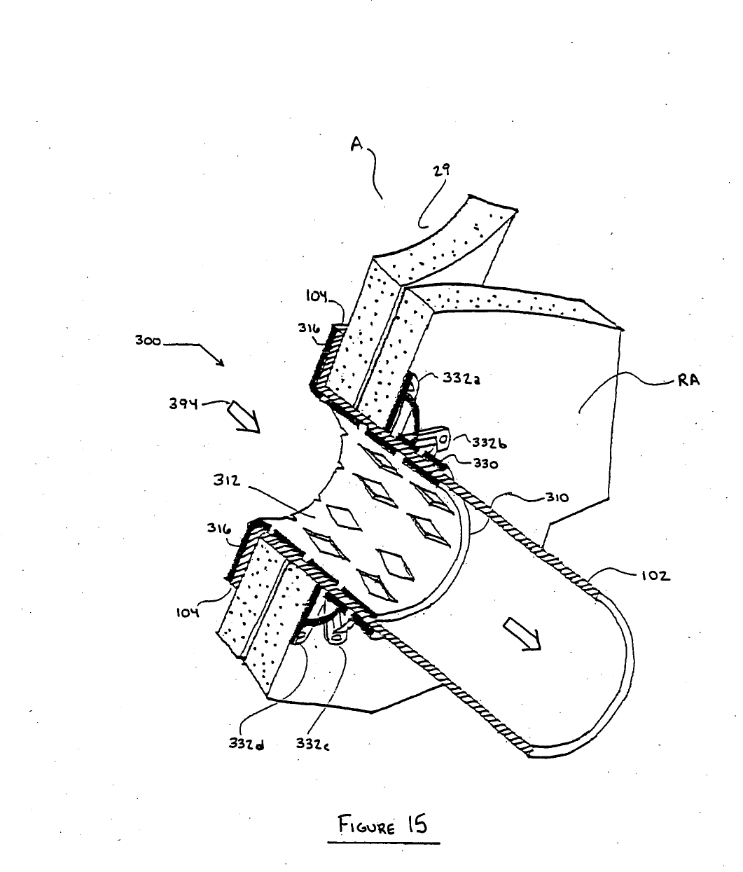

Traversing connector 300 is illustrated in FIG. 12 in a collapsed configuration for endovascular placement and in FIG. 15 in a deployed and implanted configuration in the apertures cut by the cutting member 376. Traversing connector 300 includes an inlet member 310, and a positioning member 330.

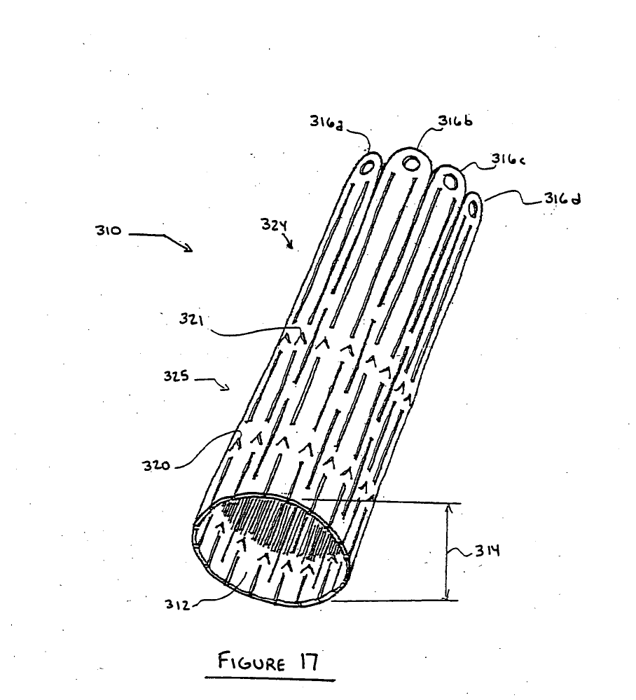

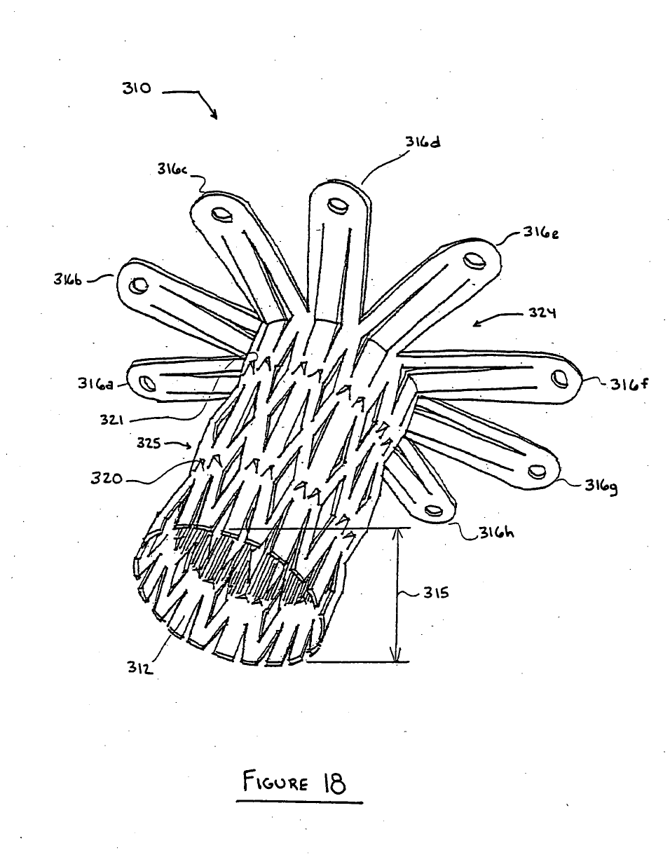

The inlet member 310 is arranged for receiving arterial blood flow from the aorta A, traversing the apertures cut by the cutting member 376, engaging an interior portion of the aorta wall, and providing the arterial blood flow to the conduit 102. Inlet member 310 includes a channel 312 for providing the arterial blood flow, and an element 316 extending radially and arranged to engage a portion of the aorta interior wall. The channel 312 may be formed by placing a portion of conduit 102 proximate to the first end 104 about an outer periphery of a portion of the inlet member 310. Additional description of the inlet member 310 is provided in conjunction with FIGS. 17 and 18.

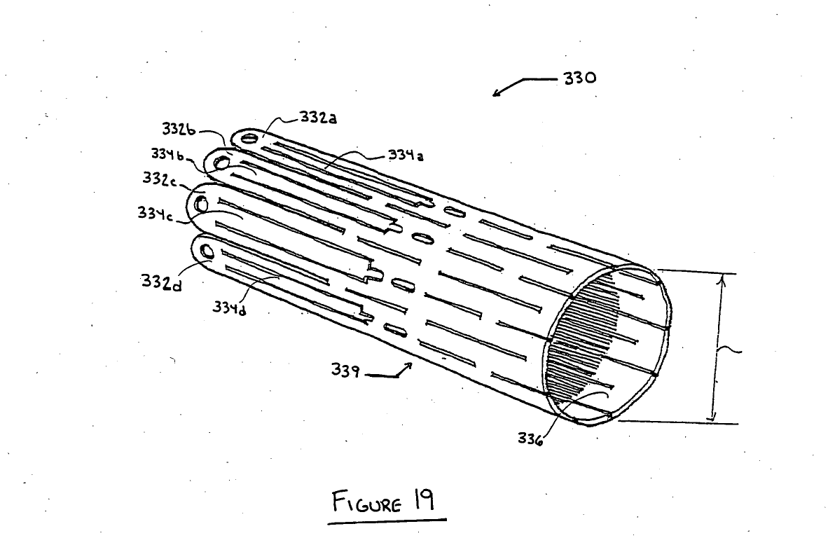

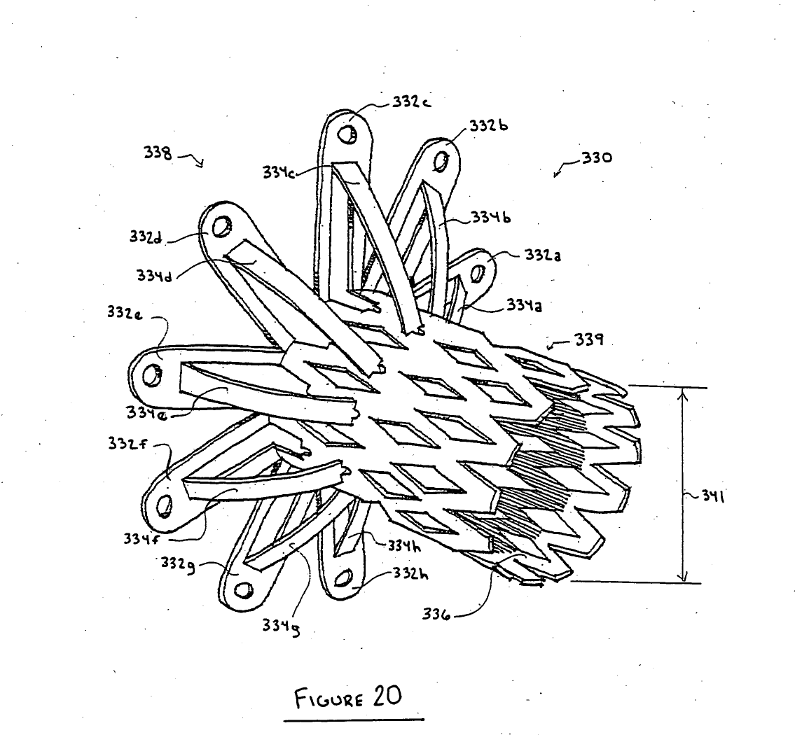

The positioning member 330 includes an interior periphery 336 arranged to engage a portion of the inlet member 310 and a portion of conduit 102 proximate to the first end 104 by resisting annular.expansion of the inlet member 310. Additional description of the positioning member 330 is provided in conjunction with FIGS. 19 and 20.

The balloon catheter 350 may be similar to the balloon catheter 250, and includes a lumen 354 arranged for following a guidewire, an expansion member 352, a cutter/deployer engaging member 359, and an elongated shaft 356 having an extracorporeal portion arranged for advancing 414 and retracting the balloon catheter 350. FIG. 12 illustrated an embodiment where the guidewire engaging member 382 is carried by the balloon catheter 250 instead of the tubular structure 372. The balloon catheter 350 may be any type of expandable catheter suitable for endovascular use, and those having a relatively short length and larger diameter may be particularly suited for use in accordance with the invention. The cutter/deployer engaging member 359 includes arrangement for transmitting advancement 414 and retraction movements of the elongated shaft 356 to the cutter/deployer 370.

Assembly 360 comprises the balloon catheter 350 coupled to the cutter deployer 370 by engaging member 359. The assembly 360 further comprises the inlet member 310 sheathed within a portion of the conduit 102 proximate to the first end 104, which is further sheathed within positioning member 330, which is further sheathed within the sheath 374 of the cutter deployer 370. When so sheathed, the inlet member 310 is arranged to exert an radially expansive force that compresses and engages the portion of conduit 102, the positioning member 330, and the sheath 374. The balloon catheter 350 may be partially expanded against the channel 312 of the inlet member 310 to provide additional radial expansive force and keep the assembly 360 together while it is advanced into the right atrium RA.

An initial step in placing the assembly 360 within the right atrium RA includes placing the extracorporeal venous end of the guidewire 410 inside the opening in guidewire following member 380, and advancing the venous end into to the lumen 354 of balloon catheter 350, thus slideably engaging the guidewire 410 in the manner described in conjunction with FIG. 8. The assembly 360 is advanced into the right atrium RA also in the manner described in conjunction with FIG. 8. The tissue-cutting member 376 may be rendered inoperative during placement of the assembly 360 in the right atrium to limit damage to vascular structures. FIG. 12 illustrates the assembly 360 advanced along guidewire 410 and adjacent to the wall of a right atrium RA at guidewire pathway 460. This position is an initial step in cutting an aperture through tissue between the right atrium RA and the aorta, and deploying the traversing connector 300.

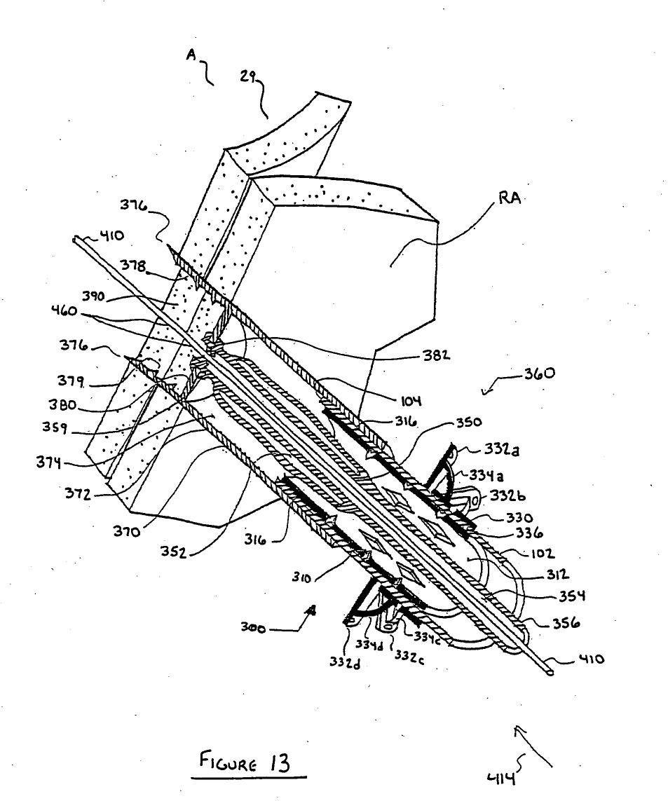

FIG. 13 illustrates intermediate steps in cutting an aperture through tissue between cavities of the right atrium RA and the aorta, and an initial step in deploying the traversing connector 300. The expansion member 352 is shown retracted for clarity in FIG. 13, but retraction at this step may not be required. An intermediate step includes partially withdrawing assembly 360 from the sheath 374 sufficient for a right atrium wall engaging element 334 to deploy in a configuration for engaging the wall of the right atrium RA and limiting advancement 414 of the inlet member 310. The engaging member 316 of inlet member 310 is prevented from expanding by its continued presence in the sheath 374.

The guidewire engaging member 382, illustrated as a pawl, is arranged to engage guidewire 410 when the extracorporeal arterial end is withdrawn a distance from the patient. Another intermediate step includes advancing the cutter/deployer 370 by moving the extracorporeal arterial end of the guidewire 410 a short distance in the advancement direction 414. This causes the engaging member 382 to engage the guidewire 410, and advance the tissue-cutting member 376 through the right atrial wall and the aortic wall. This forms apertures in the walls of the aorta A and the right atrium RA. The cutting forms cut tissue 390.

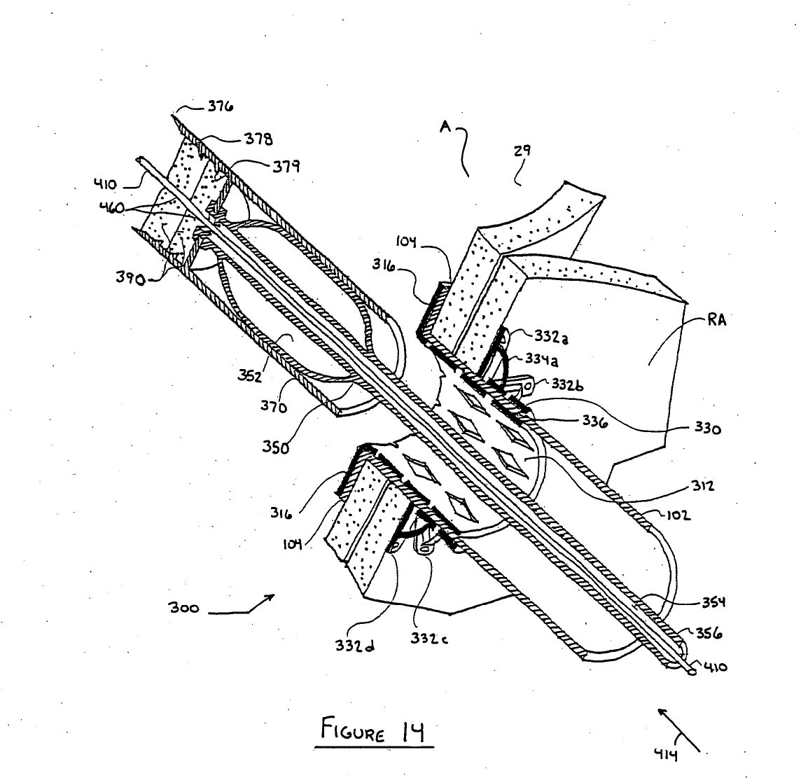

FIG. 14 illustrates another intermediate step in cutting an aperture through tissue between the right atrium RA and the aorta, and another step in deploying the traversing connector 300. As the arterial extracorporeal end of guidewire 410 is further advanced, the cutter/deployer 370 fully advances into the aorta A. An initial portion of this advancement causes the right atrium wall engaging element 330 to engage the inside of the right atrium wall, stopping further advancement of the inlet member 310.

With advancement of inlet member 310 stopped, continued advancement of tissue cutter/deployer 370 completes unsheathing the inlet member 310, and deploys the aorta wall engaging element 316. The deployment allows element 316 to move from a collapsed configuration to an expanded configuration, which includes radially extending elements 316 to engage the aorta wall. The engagement compresses the first end 104 of conduit 102 against the aorta wall as a step in forming a fluid seal. The deployment also allows the portion of the inlet member 310 located within the cut apertures to self or automatically radially expand and annularly enlarge. This compresses a portion of the conduit 102 against the apertures in the right atrium RA and the aorta A as another step in forming a fluid seal. The inlet member 310, the conduit 102, and the position member 330 are structurally connected by the radial expansion force provided by the inlet member 310. The connection may be aided or provided by barbs, hooks, or other members located on the inlet member 310 or position member 330.

In addition, the spatial relationship between the engaging element 316 and 332 is arranged such that elements 316 and 332 compress tissue of the right atrium RA and aortic A walls together as another step in forming a fluid seal and implanting the connector 300. The combined tissue thickness is approximately 2 mm.

In another embodiment, an alternative embodiment of the traversing connector 300 may be delivered over the guidewire 410 and implanted into the guidewire pathway 460 without first forming apertures in the right atrium RA and the aorta A. If some dilation of guidewire pathway 460 is required for implanting the alternative embodiment of the traversing connector 300, a mechanical dilation may precede deployment of the traversing connector 300. For example, a small balloon catheter may be advanced over the guidewire 410 and placed in the guidewire pathway 460. Inflation of the balloon will dilate the tissue surrounding guidewire pathway 460 sufficient for implantation of the alternative embodiment of inlet member 310. Alternatively, a tapered non-balloon instrument or series of such instruments could be advanced over the guidewire 410 to dilate the right atrial wall and aortic wall. The dilating apparatus may be removed before or after deployment of the traversing connector 300. Therefore, the traversing connector 300 can be delivered over the guidewire 410 with or without preparatory steps to increase the diameter of guidewire pathway 460, such as dilation or cutting an aperture between the right atrium and aorta. In a further alternative embodiment, the traversing connector 300 may be configured to include a dilating apparatus that widens the guidewire pathway 460 as traversing connector 300 is advanced over guidewire 410. In the alternative embodiment, the distal end 108 of conduit 102 may be coupled to a portion of traversing connector that extends into the right atrium RA.

FIG. 15 illustrates a final configuration of the traversing connector 300 implanted in apertures created between the right atrium RA and the non-coronary aortic sinus 29, in accordance with the invention. The inside diameter of the portion of the channel 312 of inlet member 310 located within the cut apertures is approximately 5 mm, which is greater than the approximately 4 to 4.5 mm outside diameter of the cutter/deployer 370. This allows the cutter/deployer 370 to be withdrawn back through the inlet member channel 312. A final step includes withdrawing the cutter/deployer 370 from the patient by withdrawing the balloon catheter 350 from the patient at venous introduction site 404 in a direction opposite to advancement 414. In an alternative embodiment, the cutter/deployer 370 may be withdrawn from the patient by advancement 414 until it emerges from the patient at the introduction site 402.

FIG. 16 illustrates an assembly 500 employing a knot pusher 510 for sealing the sealable exit opening 120 of arterial blood flow conduit 102, in accordance with the invention. After removal of the pushers such as balloons 250 and 350, and the guidewires 410 and 420 from the sealable exit opening 120 of the conduit 102, the sealable exit opening 120 in the tubular body of the conduit may be sealed to prevent the aortic blood flow 394 from leaking. A guidewire (410, 420) may be in left within or in proximity to the sealable exit opening 120 to aid in the positioning of a catheter 502 introduced for the purpose of sealing or plugging the aperture.

Assembly 500 includes the catheter 502, and a knot pusher 510, which may be any devices known in the art suitable for endovascular use within the heart H. FIG. 16 also illustrates sutures 520, suture post end 522, and suture loop end 524. Sutures 522 may be any suture material suitable for use with the conduit 102, and may depend on the material used for the conduit 102. In an embodiment illustrated in FIG. 16, sutures 522 were pre-placed proximal to the opening 120 prior to the conduit 102 being inserted in the patient, and the ends 522 and 524 were secured to prevent interfering with implantation of the conduit 102 in the heart H. Any suitable suture pattern may be used, including the continuous over-and-over pattern illustrated or a purse-string pattern.

Once the opening 120 is ready for closing, the distal tip 504 of catheter 502 is placed over the extracorporeal post and loop ends 522 and 524, and guided adjacent to opening 120. The distal tip 504 may be guided by a guidewire (410, 420) prior to it being removed from opening 120. Knot-tying techniques known to those in the art are used extracorporeally to create loops by looping loop end 524 around the post end 524, and using knot pusher 510 to advance the loops down the post and form knot 526.

In another embodiment, the sealable exit opening 120 includes a self-sealing device, such as a vascular introducer sheath that includes a one-way diaphragm arranged to prevent bleeding. The vascular introducer sheath will seal automatically after removal of the pushers such as balloons 250 and 350, and the guidewires 410 and 420 from the sealable exit opening 120 of the conduit 102. In a further embodiment, a prosthesis is introduced over one of the guidewires (410, 420) from the venous entry site 404 to cover or plug the sealable exit opening 120.

When all of the apparatus are removed and the sealable site opening 120 is sealed, arterial blood flow 394 will flow from the aorta A through the conduit 102 and into the coronary venous circulation towards the myocardium. The implant is a permanent means to perfuse ischemic myocardium with arterial blood from an aortic source, and does not require an open chest procedure of any kind.

FIGS. 17 and 18 are perspective views illustrating additional features of the inlet member 310, in accordance with the invention. FIG. 17 illustrates the inlet member 310 in a compressed and pre-deployment configuration. FIG. 18 illustrates the inlet member 310 in an expanded and deployed configuration, with engaging elements 316 radially extended. Inlet member 310 includes channel 312, a compressed (or collapsed) inside diameter 314, an expanded inside diameter 315, radially extending, aortic wall engaging element 316 (shown as a plurality of elements 316a-h), a first plurality of engaging members 320, optionally a second plurality of engaging members 321, and axially spaced first and second portions 324 and 325, respectively.

Inlet member 310 may be made from any material suitable for use in the heart and cardiac venous system, such as Nitinol, stainless steel, tantalum, tungsten, and platinum. Inlet member 310 may be produced by starting with a single, unitary metal tube and removing selected material until only the structure shown in FIG. 17 remains. For example, laser cutting may be used to remove material from the starting tube in order to produce inlet member 310. The tube size and any initial plastic expansion of the laser cut tube is selected to result in the inlet member 310 being radially contractible to the compressed inside diameter 314 and being self or automatically radially expandable to at least the expanded inside diameter 315.

Inlet member 310 is arranged to be annularly compressed to the compressed inside diameter 314 for placement in the sheath 374 of cutter/deployer 370. The compressed inside diameter 314 will be approximately 3.5 to 4 mm. In its expanded state, the second portion 325 is arranged to annularly enlarge to the expanded inside diameter 315. The expanded inside diameter 315 is approximately 5 mm. The inlet member 310 has an initial pre-deployment length of about 5 mm, and a material thickness of about 0.004 inches.

First portion 324 includes a first plurality of annularly spaced members 316a-h that have free end portions, and that are arranged for engaging the interior wall of aorta A. The annularly spaced members 316a-h are further arranged such, that when compressed into the sheath 374 and then deployed, they will elastically and radially move from the compressed configuration illustrated in FIGS. 12 and 17 to the expanded configuration illustrated in FIGS. 14-15, and 18, and to engage the interior wall of the aorta A.

Second portion 325 provides, a structure allowing its annular dimension to be enlarged to an expanded inside diameter 315 or reduced to the compressed inside diameter 314, and when reduced typically by compression, the structure provides an elastic force seeking to enlarge the annular dimension. Second portion 325 is particularly arranged to be radially and elastically contracted to the compressed inside diameter 314, and then to automatically and elastically radially expand upon deployment to expanded inside diameter 315. The radially expandable and contractible structure is provided by making the inlet member 310 with a plurality of annularly adjacent, annularly enlargeable portions. For example, a typical enlargeable portion includes annularly spaced, adjacent, and interconnected longitudinal members, the axially spaced ends of which are connected to one another. A portion of the longitudinal members may have free ends. A plurality of these enlargeable portions is connected side-to-side and end-to-end on second portion 325. The structure is annularly enlargeable by radial expansion, which annularly enlarges the portions, as shown for example in FIG. 18. As second portion 325 annularly enlarges, it generally axially shortens. Once the second portion 325 is plastically annularly enlarged to at least inside diameter 315, the enlargeable portions are also elastically and annularly compressible, permitting radially contracting the second portion 325 to inside diameter 314 for placement in the sheath 374.

Second portion 325 also includes a plurality of engagement facilitating members, arranged in a first band 320 and optionally a second band 321. The engagement facilitating members in bands 320 and 321 may include outward deflected material arranged to form barbs, hooks, or other shapes that facilitate coupling between the inlet member 310, the conduit 102, and the position member 370.

The outward deflection of engaging elements 316a-h, and engagement facilitating members 320 and 321 as illustrated in FIG. 18 may be produced by putting the inlet member 310 on a mandrel and plastically displacing them. In another embodiment, the inlet member 310 may be formed in such a way that second portion 325 is annularly enlargeable by inflation of a balloon catheter 350 that is temporarily disposed in the channel 312.

In use, the inlet member 310 is formed into the configuration illustrated in FIG. 18. Inlet member 310 is prepared for incorporation into assembly 360 by bringing the engaging elements 316a-h of the first portion 324 into axial alignment and by annularly compressing the second portion 325. The inlet member 310 as part of assembly 360 is then sheathed in sheath 374 for deployment. Upon deployment, the inlet member 310 deploys as illustrated in FIGS. 14-15, and 18, and engaging elements 316a-h engage the interior wall of the aorta A. The deployment further allows the second portion 325 to annularly enlarge and cause the inlet member to compressively oppose the expanded inside diameter 341 of the positioning member 330 (shown in FIG. 20). The annular enlargement provides a compressive force that fluid couples inlet member 310 and a portion of the first end 104 of the conduit 102, and further mechanically couples the second portion 325 to the positioning member 330. This annular enlargement also causes the second portion 325 to compressively oppose the tissue of the aperture formed between the right atrium RA and the aorta A.

FIGS. 19 and 20 are perspective views illustrating additional features of the positioning member 330, in accordance with the invention. FIG. 19 illustrates the positioning member 330 in a compressed and pre-deployment configuration. FIG. 20 illustrates the positioning member 330 in an expanded and deployed configuration, with engaging elements 332 and braces 334 radially extended.

The positioning member 330 is substantially similar to inlet member 310 in construction and arrangement. The positioning member 330 includes radially extending right atrium wall engaging element 332 (shown as a plurality of elements 332a-h), bracing element 334 (shown as a plurality of bracing elements 334a-h), a compressed (or collapsed) inside diameter 340, an expanded inside diameter 341, and axially spaced first and second portions 338 and 339, respectively.

The positioning member 330 may be made from the same material and made in the same manner as inlet member 310, and arranged to be compressed to the compressed inside diameter 340 for placement in the sheath 374 of cutter/deployer 370. The compressed inside diameter 340 will be approximately 3.5 to 4 mm and the expanded inside diameter 341 is approximately 5.5 mm. In its expanded state, the second portion 339 is arranged to radially expand to the inside diameter 341 and to oppose further expansion. The limitation on expansion causes the positioning member 330 to compressively oppose further expansion of the inlet member 310, cooperatively providing a compressive force coupling the second portion 325 of inlet member 310 to the second portion 339 of the positioning member 330. The compressive force also provides fluid coupling of the inlet. member 310 to a portion of the first end 104 of the conduit 102. The positioning member 330 has an initial pre-deployment length of about 5 mm, and a material thickness of about 0.004 inches.

In use, the positioning member 330 is formed into the configuration illustrated in FIG. 20. Positioning member 330 is prepared for incorporation into assembly 360 by bringing the engaging elements 332a-h and braces 334a-h into axial alignment, and by compressing second potion 339. The positioning member 330 as part of assembly 360 is then sheathed in sheath 374 for deployment. Upon deployment, the positioning member 330 deploys as illustrated in FIGS. 13-15, and 20, and engaging elements 332a-h engage the interior wall of the right atrium RA.

While the present invention has been described in certain preferred embodiments, other embodiments of the invention include an apparatus and method for providing arterial blood for arterial perfusion of ischemic myocardium. These embodiments include arrangement of the apparatus and method for implantation in a beating heart.

Although the present invention has been described in considerable detail with reference to certain preferred embodiments, other embodiments are possible. Therefore, the spirit or scope of the appended claims should not be limited to the description of the embodiments contained herein. It is intended that the invention resides in the claims hereinafter appended.

What is claimed is:

1. An assembly for use in implanting an aorta-right atrium traversing connector, the assembly comprising:
a guidewire path creation subassembly arranged for creating a guidewire pathway between an aorta and a right atrium, the subassembly including a first catheter having a distal tip arranged for placement into a cavity of a body structure and a lumen, a second catheter having a distal tip arranged for placement into a cavity of a body structure and a lumen, and a guidewire deployable from one catheter lumen and receivable by another catheter lumen and having a tissue penetrating element arranged to create a guidewire pathway by penetrating tissue between the lumens; and
a guidewire guided instrument arranged for creating an aperture in response to the guidewire pathway between the aorta and the right atrium, and deploying a connector in the aperture.

2. The assembly of claim 1, wherein the guidewire guided instrument includes a tubular sheath arranged for endovascular placement, a sheath arranged for carrying and deploying the traversing connector, a tissue-cutting element, and a guidewire following member.

3. The assembly of claim 1, wherein the guidewire guided instrument includes a movement control member for moving the instrument along a guidewire and having an extracorporeal portion.

4. The assembly of claim 1, further including a device arranged to provide arterial blood flow from the aorta to coronary venous system for venous retroperfusion of myocardium, the device including:
an aorta-right atrium traversing connector arranged to receive arterial blood flow from the aorta;
an arterial blood conduit in fluid communication with the traversing connector and a portion of the venous system, the conduit arranged for placement within the right atrium and the coronary sinus; and
a venous connector that couples the conduit to the coronary venous system.

5. The assembly of claim 1, wherein the assembly includes arrangement for use in a beating heart.

6. The assembly of claim 1, wherein the assembly includes arrangement for endovascular use.

7. An assembly for forming an aperture between an aorta and an atrium of a heart, the assembly comprising:
a guidewire placement subassembly that includes:
a first steerable catheter and second steerable catheter, each catheter arranged for placement into a cavity of a vascular structure and having a lumen with a distal end and an alignment portion proximate to the distal end, the alignment portions being arranged to align with each other such that when aligned, the distal ends are substantially aligned; and
a guidewire deployable from one distal end, receivable into the other distal end, and arranged to pass through tissue between the distal ends; and
a guidewire guidable instrument having an element arranged to form an aperture in tissue between the distal ends, such that when the distal end of one catheter is deployed in the aorta and the distal end of the other catheter is deployed in the atrium, and the guidewire is passed between the distal ends, the instrument can be guided by the guidewire through tissue between the aorta and right atrium to create the aperture.

8. The assembly of claim 7, wherein the catheters further include arrangement for transvascular placement in a vascular structure.

9. The assembly of claim 7, wherein both alignment portions are magnetic, and are arranged to attract and align with each other.

10. The assembly of claim 7, further including a penetrating element arranged to penetrate tissue between the distal ends.

11. The assembly of claim 10, wherein one catheter further includes arrangement to deploy the penetrating element and another catheter further includes an element arranged to engage the penetrating element portion.

12. The assembly of claim 10, wherein the penetrating element portion includes a penetration aid selected from a group consisting of a thermal heating element, a laser energy emitter, a RF cutting device, and a vibration device.

13. The assembly of claim 10, wherein the penetrating element portion includes a hollow needle and the guidewire is arranged for advancement through tissue penetrated by the hollow needle.

14. The assembly of claim 10, wherein the guidewire carries the penetrating element.

15. The assembly of claim 10, wherein the guidable instrument carries the penetrating element.

16. The assembly of claim 7, wherein at least one catheter further includes a substance viewable with an imaging device.

17. The assembly of claim 7, wherein at least one catheter further includes an additional lumen arranged to eject a substance viewable with an imaging device.

18. The assembly of claim 7, wherein the element of the guidewire guidable instrument includes a tissue cutter arranged to cut the aperture.

19. The assembly of claim 7, wherein the assembly further includes arrangement for use in a beating heart.

20. The assembly of claim 7, wherein the atrium is a right atrium.

21. A guidewire placement assembly comprising:
a first steerable catheter and a second steerable catheter, each catheter arranged for placement into a cavity of a vascular structure and having a lumen with a distal end and an alignment portion proximate to the distal end, the alignment portions being arranged to align with each other such that when aligned, the distal ends are substantially aligned; and
a guidewire deployable from on distal end, receivable into the other distal end, and arranged to pass through tissue between the distal ends.

22. The assembly of claim 21, wherein the catheters further include arrangement for transvascular placement in a vascular structure.

23. The assembly of claim 21, wherein one catheter includes arrangement for transvascular placement in an arterial structure and another catheter includes arrangement for transvascular placement in a venous structure.
